(19) United States
(12) Patent Application Publication
(10) Pub. No.: US 2022/0400989 A1
(43) Pub. Date: Dec. 22, 2022

(54) TREATMENT AND DIAGNOSES OF DISEASE AND MALADIES USING REMOTE MONITORING, DATA ANALYTICS, AND THERAPIES

(71) Applicant: Steven F. Myers, Rockville, MD (US)

(72) Inventor: Steven F. Myers, Rockville, MD (US)

(21) Appl. No.: 17/842,620

(22) Filed: Jun. 16, 2022

Related U.S. Application Data
(60) Provisional application No. 63/211,518, filed on Jun. 16, 2021.

(51) Int. Cl. A61B 5/1455; A61B 5/024; A61B 5/021

(52) U.S. Cl. CPC A61B 5/14551; A61B 5/02416; A61B 5/021; A61B 5/02055

(57) ABSTRACT

An example system for enhanced remote monitoring of a patient can obtain measurements of the patient's vital signs using various computer devices, such as wearable devices (e.g., smartwatch) and mobile devices (e.g., smartphone). The system implements a video-based vital sign capture function, which operates the mobile device as an optic sensor in order to obtain vital sign measurements for the patient from video imaging data of the body of the patient. The video imaging data is captured using a digital camera of the mobile device. The system can include a diagnostics server connected over a network to a data server that may host medical data silos, which is further connected to AI machine learning systems.

### RELATED APPLICATIONS

Under provisions of 35 U.S.C. § 119(e), the Applicant claims the benefit of U.S. provisional application no. 63/211,518, filed Jun. 16, 2021, which is incorporated herein by reference.

### TECHNICAL FIELD

This disclosure generally relates to novel treatments and diagnoses of health issues. More specifically, this disclosure relates to treatment and diagnoses of disease and maladies using remote monitoring, data analytics, and therapies.

It is intended that each of the referenced applications may be applicable to the concepts and embodiments disclosed herein, even if such concepts and embodiments are disclosed in the referenced applications with different limitations and configurations and described using different examples and terminology.

### BACKGROUND

Despite advances in prevention, treatment, and management of diseases and maladies, racial disparities persist. For example, for cardiovascular diseases (CVD), the CVD accounts for one-third of differences in mortality rates in racial minorities compared to whites. The non-whites experience the highest mortality rates. Further complicating CVD outcomes, anxiety and depression often co-occur with and are known risk factors for the CVD, doubling the chances a patient will die from the CVD. Evidence shows that the CVD patients with untreated anxiety and/or depression experience poorer outcomes, including hospitalizations because of major adverse cardiovascular events (MACEs). For instance, increased prevalence of depression in patients with CVD is associated with a significant increase in hospitalization and death. Such phenomena have been explained by the fact that several pathological mechanisms underlying depression and anxiety are in line with pathological mechanisms of the CVD, such as elevated platelet activities, autonomic and immune dysregulation, elevated inflammatory process, metabolic abnormalities, and accumulation of oxidative stress.

It is not known if the link between CVD and depression/anxiety is independent of comorbid medical diseases, or if these associations depend on race and ethnicity. From a treatment perspective, antidepressants have not been shown to be effective for preventing MACE's in the CVD patients with depression/anxiety. Innovative treatments are needed, especially so, in non-whites with the goal of improving access and reducing barriers to care. Evidence-based telemedicine tools have shown efficacy when used alone and could be integrated to improve outcomes for patients with the CVD and depression/anxiety, with an emphasis on interventions designed to address barriers to care typically experienced by racially diverse, non-white patients.

Accordingly, a system and method for prediction and diagnosis of depression and/or anxiety in non-white CVD patients using an artificial intelligence-based model are desired. This novel system and method have various applications for prediction and diagnosis of various diseases and maladies for all patient and consumer populations using unique artificial intelligence-based models and neural network algorithms.

### SUMMARY

This disclosure relates to treatment and diagnoses of disease and maladies using remote monitoring, data analytics, and behavioral therapies. Remote monitoring of a patient can involve a distinct video-based vital sign capture capability, which utilizes a mobile device (having a digital camera embedded therein) as an optical sensor that can measure vital signs of the patient. Video-based vital sign capture can involve capturing video imaging data of one or more body areas of a patient, wherein the video imaging data is captured using a mobile device. Further, analyzing the video imaging data using one or more optical analysis techniques, where these techniques include color-based analysis and/or motion-based analysis of the video imaging data. Additionally, generating one or more waveforms based on the one more optical analysis techniques, wherein the one or more waveforms indicate physiological changes associated with each of the one or more body areas of the patient to represent vital signs of the patient. The measurements of the vital signs of the patient are determined based on the generated one or more waveforms.

In one or more instances, this disclosure may relate to minority health issues. In at least one instance, this disclosure may relate to the use of remote monitoring, statistical models, behavioral therapies/analysis, and/or data analytics of various populations (including minority and/or other underserved populations). In at least on instance, this disclosure may relate to cardiovascular patients and the use of remote monitoring, statistical models, behavioral analysis, data analytics for prediction and diagnosis of anxiety and depression using an artificial intelligence-based model. More specifically, this may be used with regards to treatment and diagnoses of anxiety and depression in cardiovascular patients among other maladies and diseases.

A variety of predictive analytics can be run using various statistical models from data collected from IoT devices (e.g., wearable devices, electronic tattoos, medical diagnostic devices, MRI, CT Scanners, Ultrasound, etc.). For example, while implantable cardiac sensors have been effective at reducing hospitalization for heart failure, wearable technology and noninvasive approaches including remote monitoring may be used with the predictive analytics system to predict heart failure rehospitalization. This system may utilize multi-sensor noninvasive remote monitoring for prediction of heart failure exacerbation. In one or more instances this novel remote monitoring system for diagnoses and predictive health outcomes can be used with various IoT devices (e.g., wearable devices, electronic tattoos, medical diagnostic devices, MRI, CT Scanners, Ultrasound, etc.) to predict mortality, readmission, and/or emergency department visits.

In one or more instances this novel remote monitoring system for diagnoses and predictive health outcomes can be used with various IoT devices (e.g., wearable devices, electronic tattoos, medical diagnostic devices, MRI, CT Scanners, Ultrasound, etc.) to improve quality of care, quality of life, minimize unnecessary invasive surgeries, prevent complications, aid in falls prevention, treat life-threatening situations, provide for urgent care interventions for certain categories of chronic patients. In one or more instances this novel remote monitoring system for diagnoses and predictive health outcomes can be used with various IoT devices (e.g., wearable devices, electronic tattoos, medical diagnostic devices, MRI, CT Scanners, Ultrasound, etc.) to improve the quality of home care, nursing care treatment, provide various means for quality control of health professionals, provide either real-time and/or continuous monitoring of patients with chronic conditions.

For the purposes of example and to give a detailed description for the following disclosure, the case of utilizing this unique system and method for prediction and diagnosis of diseases and maladies for patient populations using unique artificial intelligence-based models and neural network algorithms and will be demonstrated using the example cardiovascular disease (CVD) patients from a non-white or underserved, minority community. As such, what follows is a description of a system and method for prediction and diagnosis of depression and/or anxiety in non-white CVD patients using an artificial intelligence-based model including neural network algorithms.

Cognitive behavioral therapy (CBT, known as iCBT when delivered remotely via the Internet) reduces anxiety and depression in CVD patients. Despite this evidence, the CBT is not generally incorporated into clinical management of the CVD patients with anxiety and/or depression. This gap is likely due to issues such as a fragmented health care system, lack of integrated technology solutions, lack of health insurance, and shortage of mental health professional capacity—particularly during the pandemic—that often disproportionately impacts access to mental health services for non-white, marginalized individuals.

Remote patient monitoring (RPM)—also delivered via the internet—is a standard of care for the CVD patients with recent hospitalization. The RPM is a digital intervention that has shown to improve clinical management and outcomes for the CVD patients, and therefore represents a vehicle onto which can be layered additional interventions such as iCBT. The RPM may include activity from wearable devices. Virtual visits, relied on during the COVID-19 pandemic, remove care barriers for vulnerable patients and have been shown to be preferred by patients with depression and anxiety. Although racial disparities persist in the use and outcomes of telemedicine, research suggests telemedicine interventions designed with input from non-white patients can reduce racial disparities. To date, only one study examined the prevalence of depression and/or anxiety by race/ethnicity in CVD patients. None have examined feasibility of combining iCBT, RPM, activity from wearable devices (i.e., sleep; steps; temperature; respiration; heart rate variability; resting heart rate; floors climbed; heart rhythm assessment; oxygen saturation; stress management tools EDA sensor; heart rhythm assessment) and virtual visits into a single intervention to improve outcomes.

As discussed above, persistent racial disparities remain for patients with the CVD and are exacerbated with co-occurring depression and/or anxiety. Little is known about racial differences with regard to prevalence of depression and anxiety among CVD patients. It is also not known whether a link between CVD and depression/anxiety is independent of race or comorbidity. Furthermore, it is unclear which effective treatments may improve outcomes for particular types of population. Currently, there is no method or system that can implement and disseminate an evidence-based intervention that improves outcomes for all CVD patients and reduces health disparities for non-white CVD patients with depression/anxiety.

A diagnostics server may be connected over a network to a data server that may host medical data silos. The diagnostics server may be connected to remote users (such as doctors or patients) over the network. The diagnostics server may be connected to AI machine learning systems. The diagnostics server may provide training data from a local data source or form a blockchain ledger to train the models of the AI machine learning systems. The AI machine learning system(s) may be trained with the output of the data source, neural networks or the blockchain. One example embodiment provides a processor and memory of a diagnostics server, wherein the processor is configured to execute instructions to provide data to the AI system and to process the predicted diagnosis data. The present disclosure may provide an AI remote patient monitoring system configured to execute instructions to provide data to the AI system and to process the predicted diagnosis data. The present disclosure may provide an AI remote patient monitoring platform configured to execute instructions to provide data to the AI system and to process the predicted diagnosis data. The present disclosure includes many aspects and features. Moreover, while many aspects and features relate to, and are described in, the context of methods, techniques, and systems for providing AI assisted remote patient monitoring, data collection, data science and analysis. While the context of the present disclosure includes a focus on data collection and treatment of underrepresented minorities and non-white CVD patients, the applications of the technological functions described herein are not limited to that patient group, focus, or data set. There are many other applications including but not limited to economic and commercial applications of AI assisted remote consumer monitoring including data collection, data science and analysis.

### BRIEF DESCRIPTION OF THE DRAWINGS

FIG. 1 illustrates how the CVD patients' data is collected from variety of sources including wearable devices, according to example embodiments.

FIG. 2A illustrates a network diagram of a system including an AI module and a model database, according to example embodiments.

FIG. 2B illustrates a network diagram of a system including an AI module and a blockchain, according to example embodiments.

FIG. 3 illustrates an example of a blockchain which stores machine learning AI data, according to example embodiments.

FIG. 4 illustrates an example server system that supports one or more of the example embodiments.

FIG. 5 illustrates an example of a neural network architecture used for storing and mapping relations of input-output model data.

FIG. 6 illustrates an example of a neural network architecture used for storing and mapping relations of input-output model data.

FIG. 7 illustrates an example of a deep learning neural network architecture used for storing and mapping relations of input-output model data.

FIG. 8A illustrates assessment tools, Generalized Anxiety Disorder (GAD)-7 and Patient Health Questionnaire (PHQ)-9 screening tools, according to example embodiments.

FIG. 8B illustrates Social Determinants of Health (SDOH) factors that can be used as data for assessment tools, datasets and predictive analytics utilized within the system including an AI module and a blockchain, according to example embodiments.

FIG. 8C illustrates a Social Determinants of Health (SDOH) Flowchart with factors that can be used as data for assessment tools, datasets and predictive analytics utilized within the system including an AI module and a blockchain, according to example embodiments.

FIG. 9 depicts an example of a system architecture for an eMedical Sentry system implementing remote monitoring of a patient, according to example embodiments.

FIG. 10 depicts an example of optical/image analysis techniques utilized to implement a video-based vital sign capture feature of the system shown in FIG. 9, according to example embodiments.

FIG. 11 depicts examples of graphical user interfaces (GUI) that may be generated by a software application (app) associated with the system shown in FIG. 9, according to example embodiments.

FIG. 12 depicts an example of a GUI that may be generated by a software application (app) associated with the system shown in FIG. 9, according to example embodiments.

FIG. 13 illustrates an example deployment scenario for various elements of the system shown in FIG. 9, according to example embodiments.

FIG. 14 depicts an operational example of remotely monitoring vital signs of a patient using the video-based vital sign capture feature of the system shown in FIG. 8, according to example embodiments.

FIG. 15 illustrates an example computer system executing a process to implement the video-based vital sign capture capabilities, according to example embodiments.

FIG. 16 illustrates an example computer system, which may implement any of the embodiments of the present invention.

### DETAILED DESCRIPTION

It will be readily understood that the instant components, as generally described and illustrated in the figures herein, may be arranged and designed in a wide variety of different configurations. Thus, the following detailed description of the embodiments of at least one of a method, apparatus, non-transitory computer readable medium and system, as represented in the attached figures, is not intended to limit the scope of the application as claimed but is merely representative of selected embodiments.

The instant features, structures, or characteristics as described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

In addition, while the term “message” may have been used in the description of embodiments, the application may be applied to many types of network data, such as, packet, frame, datagram, etc. The term “message” also includes packet, frame, datagram, and any equivalents thereof. Furthermore, while certain types of messages and signaling may be depicted in exemplary embodiments they are not limited to a certain type of message, and the application is not limited to a certain type of signaling.

Example embodiments provide methods, systems, components, non-transitory computer readable media, devices, and/or networks, which provide for remote monitoring of non-white cardiovascular patients for prediction and diagnosis of anxiety and depression using artificial intelligence-based models.

The exemplary embodiments are focused on better understanding prevalence and links between CVD, depression and/or anxiety, and race, as well as on testing an intervention to address disparities in non-white CVD patients with depression and/or anxiety. The central hypothesis is that non-white CVD patients have higher rates of depression/anxiety compared to the general population, and therefore may benefit from a novel, integrated intervention in accordance to the exemplary embodiments. The rationale underlying the proposed approach is three-fold. There is simultaneously a clear need to fill gaps in knowledge around prevalence and reducing disparities in non-white CVD patients with depression and/or anxiety, and a need to develop effective interventions to improve outcomes for this population. Finally, remote patient monitoring (RPM), and virtual visits are evidence-based approaches to managing CVD and internet-based cognitive behavioral therapy (iCBT) has been shown to be effective for depression and/anxiety, but no studies have tested the integration of the three to impact outcomes for CVD patients (of any race) with depression and/or anxiety.

According to exemplary embodiments, the central hypothesis may be tested by pursuing three specific aims: 1) using mixed method approach to document baseline characteristics/prevalence/associations in real world settings and use community-based participatory research (CBPR) methods to better understand needs of non-white patients with CVD and depression and/or anxiety; and 2) develop, test, validate and evaluate a novel, evidence-based, remote intervention to reduce health disparities for high-risk CVD patients with depression and/or anxiety; and 3) provide for conduction of randomized controlled trials (RCTs) to assess acceptability, feasibility, and efficacy of the intervention, as wells as apply methods and procedures to plan a subsequent fully-powered RCT.

The exemplary embodiments may pursue these aims by collecting and analyzing data on patients with CVD and depression and/or anxiety from four cardiology practices affiliated with major health systems across the country and provide and test an intervention to impact outcomes. The proposed intervention may test an innovative combination of RPM, iCBT, and virtual visits—into a seamless intervention for CVD patients with depression and anxiety. The exemplary embodiments have the potential for filling a critical gap in care and elevating the standard of care for non-white CVD patients with depression/anxiety. The results will have a positive impact by establishing a better understanding of the CVD and depression and/or anxiety among non-white patients and lay the groundwork for novel treatment approaches to eliminate disparities among this population. In addition, to improving care for the CVD patients, the results will have broader impact, contributing to understanding of remote care delivery to non-white patients during the COVID-19 pandemic, and the potential for digital care delivery to reduce access barriers for populations experiencing health disparities.

In one embodiment, three evidence-based modalities—iCBT, RPM (including wearable devices) and virtual visits—may be combined into a seamless, intuitive, tablet-based intervention for the CVD patients with depression and anxiety filling a critical gap in evidence and elevating the standard of care. The exemplary embodiments may implement and disseminate evidence-based interventions to improve outcomes and reduce health disparities for non-white CVD patients with depression/anxiety. The feasibility of the proposed approach is supported by: 1) early preliminary work and expertise of the study PI and partners; 2) a clear need to better understand prevalence and associations of CVD patients with depression and/or anxiety; and 3) the large evidence-base for RPM in reducing hospital readmission rates and for iCBT in reducing the rates of depression and anxiety, suggesting that integrating the three interventions may be effective.

The exemplary embodiment may use Deep Learning models for detection of bio-markers, demographic and psychometric data associated with depression and/or anxiety in CVD patients. In one embodiment, an artificial intelligence (AI), machine learning (ML) systems may be employed for detection or prediction of depression and/or anxiety in CVD patients.

Machine learning relies on vast quantities of historical data (or training data) to build predictive models for accurate prediction on new diagnosis-related data. According to one exemplary embodiment, machine learning analytics using data from wearable sensors can be used to more accurately predict depression and/or anxiety in CVD patients. The study shows that wearable sensors coupled with machine learning analytics have predictive accuracy comparable to implanted devices with respect to depression and/or anxiety in CVD patients. The AI system, in accordance with the exemplary embodiment, may provide a basis for prospective testing of the clinical efficacy of the proposed data-driven approach to improve clinical outcomes in detection of depression and/or anxiety in CVD patients.

Machine learning software may sift through millions of records to unearth non-intuitive patterns. In the example embodiment, a diagnostics platform may build and deploy a machine learning model for predictive monitoring and detection of depression and/or anxiety in CVD patients based on diverse data collected by medical institutions remotely. The diagnostics platform may be a cloud platform, a server, a web server, a personal computer, a user device attached to the AI system, and the like. A neural network or blockchain may be used to improve both a training process of the machine learning model and a predictive process based on a trained machine learning model. For example, rather than requiring a data scientist or a doctor or other user to constantly collect new data, historical data may be stored on neural network or on the blockchain. This can significantly reduce the collection time needed by the diagnostics platform when performing predictive model training.

Accordingly, the exemplary embodiments provide for a specific solution to a problem in the field of remote diagnosis and prediction/detection of depression and/or anxiety in CVD patients. According to the exemplary embodiments, a method, system and a computer readable medium for prediction and diagnosis of anxiety and depression in CVD patients using artificial intelligence-based models are provided.

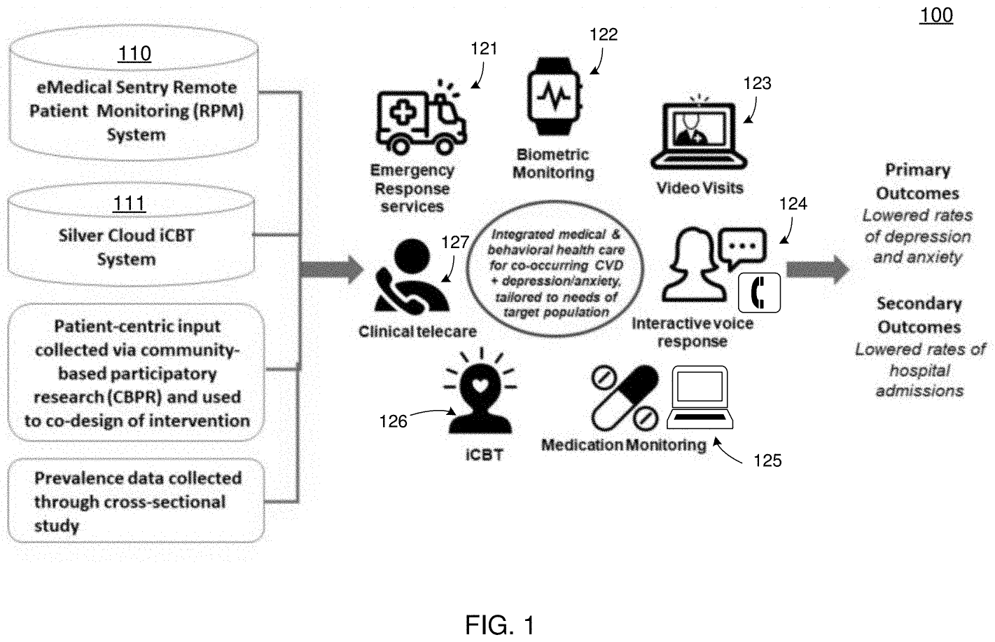

FIG. 1 illustrates an example of a system 100 that enables CVD patients' data to be collected from variety of sources. As seen in FIG. 1, the system 100 includes the integration of several communication points that can serve as the basis for collecting and/or receiving patient data, including: emergency response services illustrated as a medical emergency vehicle (e.g., ambulance) 121; biometric monitoring illustrated as a wearable device 122; video communication (e.g., video patient visits, medical appointments) illustrated as a laptop computer 123; interactive voice response (IVR) illustrated as an IVR device (e.g., telephone) 124; medical monitoring illustrated as a prescription/medicine distribution center (e.g., pharmacy) computer device 125; an iCTB 126; and clinical telecare illustrated as a telephony device (e.g., telephone) 127. The system 100 also includes an eMedical Sentry Remote Patient Monitoring System 110; and a silver cloud iCBT system 111. For example, the system 100 can be employed to collect patient-centric input via community based participatory research (CBPR) and used to co-design an intervention for a patient. Additionally, prevalence data may be collected through cross-sectional study.

The integration depicted in FIG. 1 may enable cardiologists to digitally screen patients for depression and/or anxiety using the validated Generalized Anxiety Disorder (GAD)-7 and Patient Health Questionnaire (PHQ)-9 screening tools. GAD-7 (Generalized Anxiety Disorder) is a 7-question screening tool that identifies whether a complete assessment for anxiety is indicated. PC-PTSD is a four-item screen designed for use in primary care and other medical settings to screen for post-traumatic stress disorder. Patient Health Questionnaire (PHQ)-9 is a self-administered version of the PRIME-MD diagnostic instrument for common mental disorders. The PHQ-9 is the depression module, which scores each of the nine DSM-IV criteria as “0” (not at all) to “3” (nearly every day). It has been validated for use in primary care. It is not a screening tool for depression but it is used to monitor the severity of depression and response to treatment. However, it can be used to make a tentative diagnosis of depression in at-risk populations—e.g., those with coronary heart disease or after stroke. When screening for depression the Patient Health Questionnaire (PHQ-2) can be used first (it has a 97% sensitivity and a 67% specificity). If this is positive, the PHQ-9 can then be used, which has 61% sensitivity and 94% specificity in adults. Validity has been assessed against an independent structured mental health professional (MHP) interview. PHQ-9 score ≥10 had a sensitivity of 88% and a specificity of 88% for major depression. It can even be used over the telephone.

If patients screen positive (>=10 for PHQ-9 and >=8 for GAD-7), then they may be referred to the Silver Cloud iCBT program via the eMedical Sentry platform, where the patients may have access to a full complement of remote patient monitoring (RPM) functionalities.

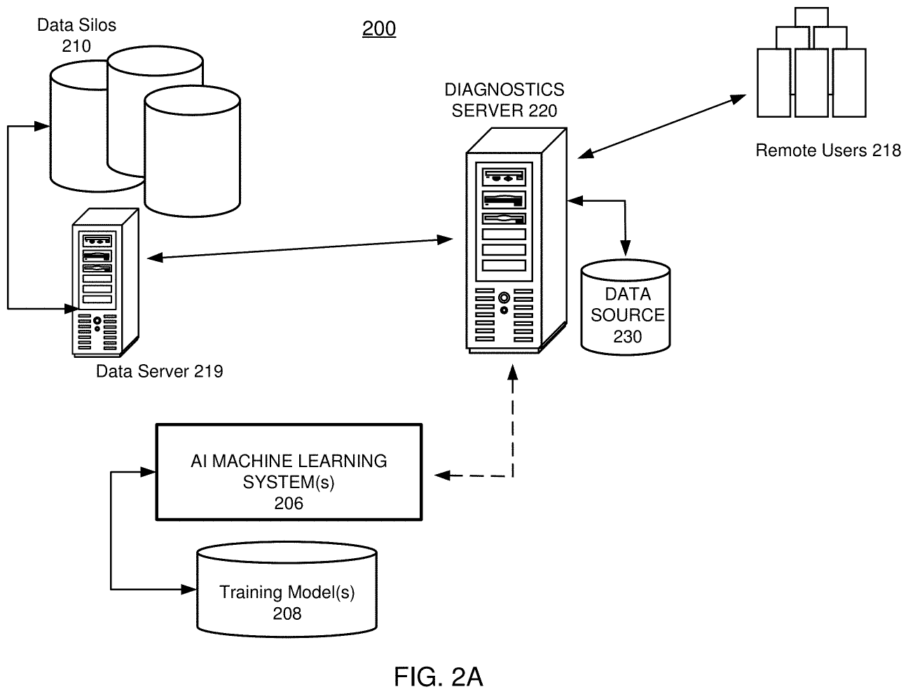

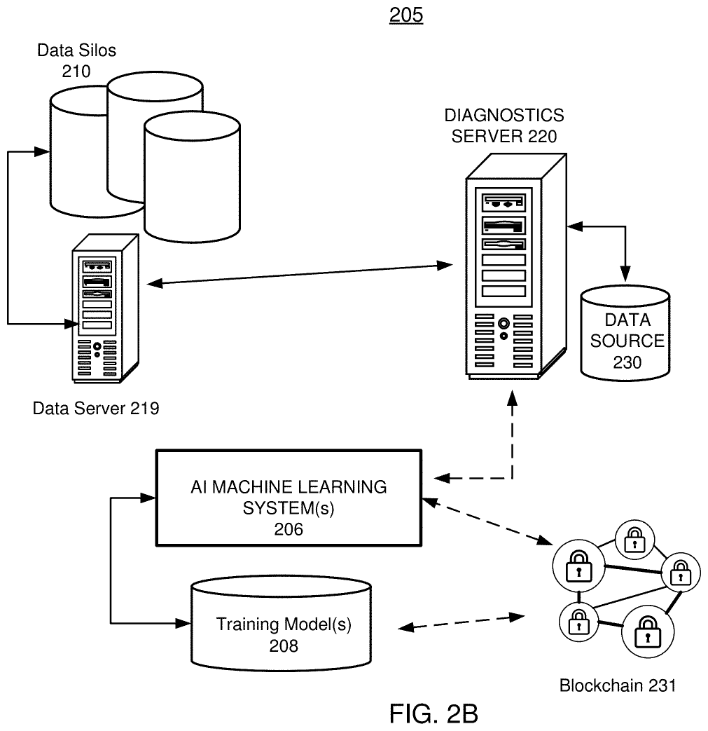

FIG. 2A illustrates a network diagram 200 of a system including an AI module and a model database, according to example embodiments. Referring to FIG. 2A, a diagnostics server 220 may be connected over a network to a data server 219 that may host medical data silos. The diagnostics server 220 may be connected to remote users (such as doctors or patients) 118 over a network. In one embodiment, the diagnostics server 220 may be connected to AI machine learning systems 206. The diagnostics server 220 may provide training data from a local data source 230 to train models 208 of the AI machine learning systems 206. The AI machine learning system(s) 206 may be trained with the output of the data source 230 (e.g., U-Net). The diagnostics server 220 may provide training data to train models of the AI machine learning systems 206.

While this example describes in detail only one diagnostics server 220, multiple such nodes may be connected to the AI machine learning system(s) 206. It should be understood that the diagnostics server 220 may include additional components and that some of the components described herein may be removed and/or modified without departing from a scope of the diagnostics server 220 disclosed herein. The diagnostics server 220 may be a computing device or a server computer, or the like, and may include a processor, which may be a semiconductor-based microprocessor, a central processing unit (CPU), an application specific integrated circuit (ASIC), a field-programmable gate array (FPGA), and/or another hardware device. Although a single processor is typically used, it should be understood that the diagnostics server 220 may include multiple processors, multiple cores, or the like, without departing from the scope of the diagnostics server 220 system.

The diagnostics server 220 may also include a non-transitory computer readable medium that may have stored thereon machine-readable instructions executable by the processor. Examples of the non-transitory computer readable medium may include an electronic, magnetic, optical, or other physical storage device that contains or stores executable instructions. For example, the non-transitory computer readable medium may be a Random-Access memory (RAM), an Electrically Erasable Programmable Read-Only Memory (EEPROM), a hard disk, an optical disc, or other type of storage device. The processor 104 may fetch, decode, and execute the machine-readable instructions to collect data from the silos and to provide the collected data and the learning data sets to the AI machine learning systems 206 to produce diagnosis or predictions.

FIG. 2B illustrates a network diagram 205 of a system including an AI module and a blockchain, according to example embodiments. Referring to FIG. 2B, a diagnostics server 220 may be connected over a network to a data server 219 that may host medical data silos. In one embodiment, the diagnostics server 220 may be connected to AI machine learning systems 206. As discussed above with reference to FIG. 2A, the diagnostics server 220 may provide training data from a local data source 230 to train models 208 of the AI machine learning systems 206. In the embodiment depicted in FIG. 2B, the diagnostics server 220 may provide the training data from a ledger of a blockchain 231 to train the models 208 of the AI machine learning systems 206. The AI machine learning system(s) 206 may be trained with the training data sets retrieved from the blockchain 231.

Referring to FIG. 2B, a diagnostics server 220 may be connected over a network to a data server 219 that may host medical data silos. In one embodiment, the diagnostics server 220 may be connected to AI machine learning systems 206. As discussed above with reference to FIG. 2A, the diagnostics server 220 may provide training data from a local data source 230 to train models 208 of the AI machine learning systems 206. In the embodiment depicted in FIG. 2B, the diagnostics server 220 may provide the training data from a ledger of a blockchain 231 to train the models 208 of the AI machine learning systems 206. The AI machine learning system(s) 206 may be trained with the training data sets retrieved from the blockchain 231.

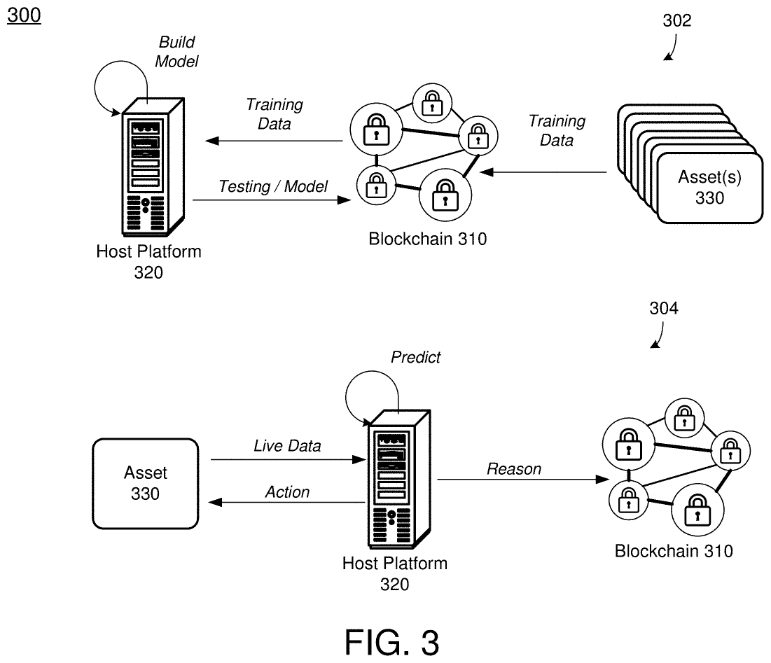

FIG. 3 illustrates an example 300 of a blockchain 310 which stores machine learning (AI) data. Machine learning relies on vast quantities of historical data (or training data) to build predictive models for accurate prediction on new data. Machine learning algorithm may sift through millions of records to unearth non-intuitive patterns based on data retrieved from neural networks or other sources.

In the example depicted in FIG. 3, a host platform 320 builds and deploys a machine learning model for predictive monitoring of assets 330. Here, the host platform 320 may be a cloud platform, an industrial server, a web server, a personal computer, a user device, and the like. Assets 330 can represent patient medical data and other patient parameters such as race, previous diagnosis, etc.

The blockchain 310 can be used to significantly improve both a training process 302 of the machine learning model and a column separation predictive process 304 based on a trained machine learning model. For example, in 302, rather than requiring a data scientist/engineer or other user to collect the data, historical data may be stored by the assets 330 themselves (or through an intermediary, not shown) on the blockchain 310. This can significantly reduce the collection time needed by the host platform 320 when performing predictive model training. For example, using smart contracts, data can be directly and reliably transferred straight from its place of origin (e.g., from a pipeline monitoring utility) to the blockchain 310. By using the blockchain 310 to ensure the security and ownership of the collected data, smart contracts may directly send the data from the assets to the individuals that use the data for building a machine learning model. This allows for sharing of data among the assets 330.

The collected data may be stored in the blockchain 310 based on a consensus mechanism. The consensus mechanism pulls in (permissioned nodes) to ensure that the data being recorded is verified and accurate. The data recorded is time-stamped, cryptographically signed, and immutable. It is therefore auditable, transparent, and secure. Adding IoT devices (e.g., wearable devices, electronic tattoos, medical diagnostic devices, MRI, CT Scanners, Ultrasound, etc.) which write directly to the blockchain can increase both the frequency and accuracy of the data being recorded.

Furthermore, training of the machine learning model on the collected data may take rounds of refinement and testing by the host platform 320. Each round may be based on additional data or data that was not previously considered to help expand the knowledge of the machine learning model. In 302, the different training and testing steps (and the data associated therewith) may be stored on the blockchain 310 by the host platform 320. Each refinement of the machine learning model (e.g., changes in variables, weights, etc.) may be stored on the blockchain 310. This provides verifiable proof of how the model was trained and what data was used to train the model. Furthermore, when the host platform 320 has achieved a finally trained model, the resulting model data may be stored on the blockchain 310.

After the model has been trained, it may be deployed to a live environment where it can make leak-related predictions/decisions based on the execution of the final trained machine learning model. In this example, data fed back from the asset 330 may be input into the machine learning model and may be used to make diagnosis/predictions such as depression/anxiety in the CDV patients. Determinations made by the execution of the machine learning model (e.g., depression/anxiety diagnosis, etc.) at the host platform 320 may be stored on the blockchain 310 to provide auditable/verifiable proof. As one non-limiting example, the machine learning model may predict a future diagnosis to a part of the asset 330 and create alert or a notification to the CDV patient. The data behind this decision may be stored by the host platform 320 on the blockchain 310. In one embodiment, the features and/or the actions described and/or depicted herein can occur on or with respect to the blockchain 310.

Machine learning algorithms may sift through millions of records to unearth non-intuitive patterns based on data retrieved from neural networks or other sources. Neural networks—their structure was inspired by the structure of the human brain, and its particular neurons by the structure of human nerve cells, although looking for far-reaching analogies with the human mind does not make much sense, as we still do not fully understand the workings of the human brain. In neural networks, subsequent neurons transmit the input signal deeper and deeper, modifying it according to the “weights” they are assigned. A neural network is capable of learning weights in a process called training, based on the sample data it receives, such as images. Interestingly, deep neural networks do not only ‘remember’ training data, but they are also very effective at ‘generalizing’, so they can deal with data they have never seen before.

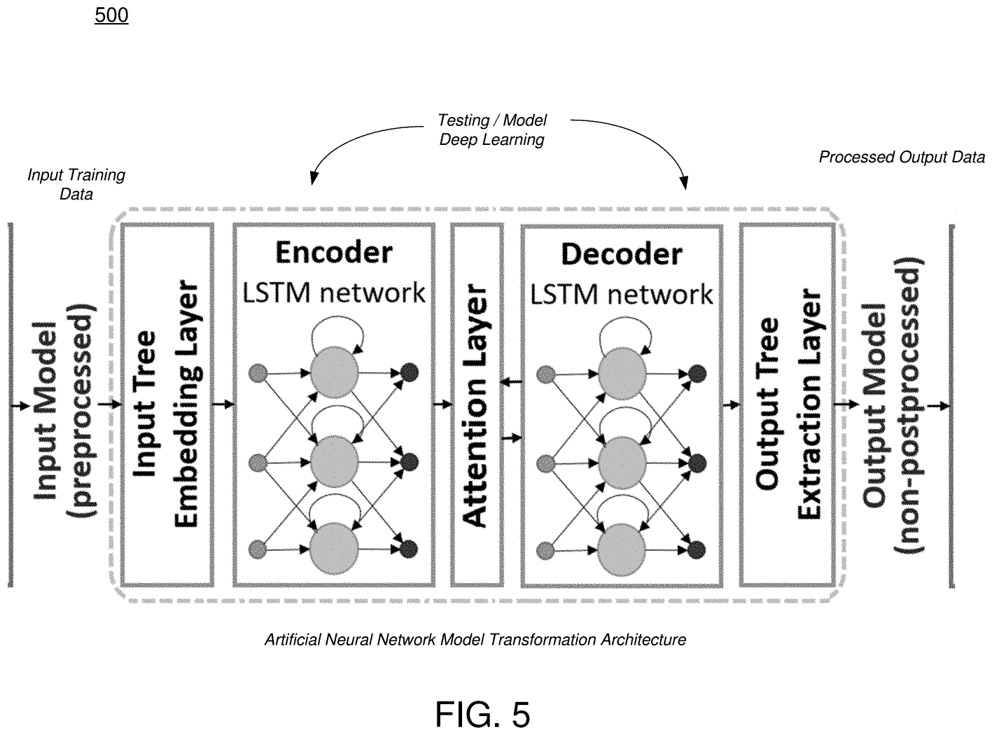

FIG. 5 illustrates an artificial neural network (ANN) model transformation architecture used for storing and mapping relations of input-output model data. All the components of the architecture have certain parameters that need to be configured to optimize the training phase depending on the specific problem they are going to solve, in our case which ailments, maladies or diseases data need to be analyzed, predicted, or diagnosed based on the data received and being input into the systems from wearables or other input data. Based on the type of assessment or analysis required, the core data values including the number of layers in each ANN, the number of neurons in each layer, the initial connection weights, the learning rate, the decay, the optimization algorithm, the dropout rate, and the attention mechanism can be set for the neural network model.

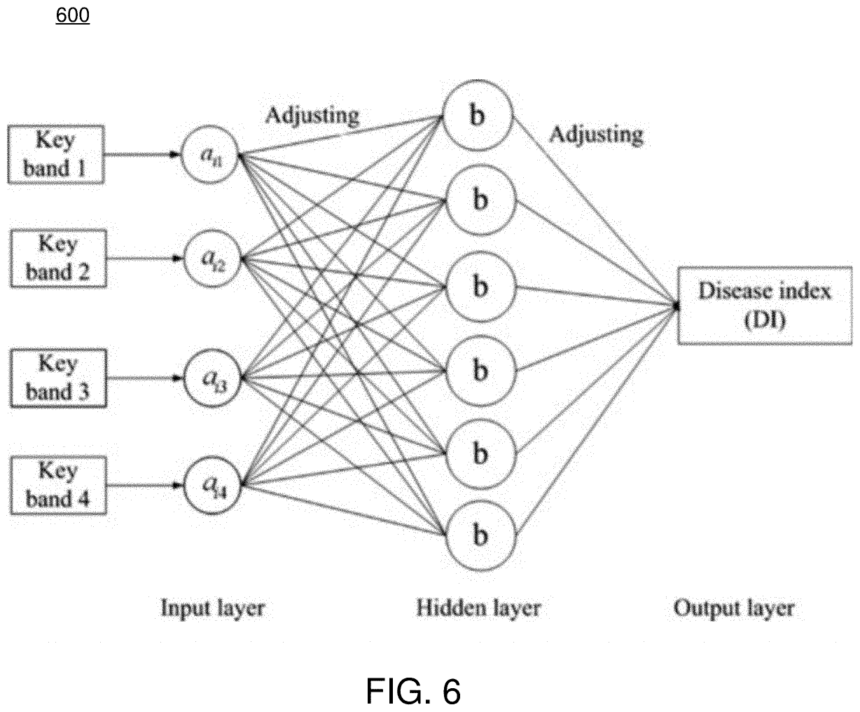

FIG. 6 illustrates an example of a back propagation neural network (BP-neural network) architecture used for storing and mapping relations of input-output model data. A BP-neural network model is a full-connected neural network including input layer, hidden layer, and output layer. The goal of the training process for a BP-neural network may be to find the weights that minimize some overall error measures, such as the sum of squared errors (SSE) or mean squared errors (MSE). Hence, the neural network training or learning utilized with this type of neural network is actually one that minimizes specific error measures. In this study, a three-layer neural network is applied in rice neck blast modeling. First, choose initial values of the parameters of the network (i.e., the connection weights and the neuron residual error values). Second, four key wavebands were selected as inputs, and grade of severity (DI values) were selected as outputs. Third, the network is trained and tested with the Matlab 6.5 software (Ju et al. 2009).

A total of 155 samples were collected, and 105 samples, or two-thirds of the total samples, were used as a training dataset to develop prediction models for disease severity. The remaining 50 samples, or one-third of the total samples, were used as a test dataset. The samples were not randomly split into these two sets because this could have resulted in sets with samples that were not representative of the overall distribution of disease severity sampled. Therefore, the samples were arranged in order of disease severity. Every third sample was separated into the validation set, and the remaining samples comprised the calibration set. This split ensured that both data sets had samples with similar distributions of disease severity (Jones et al. 2010).

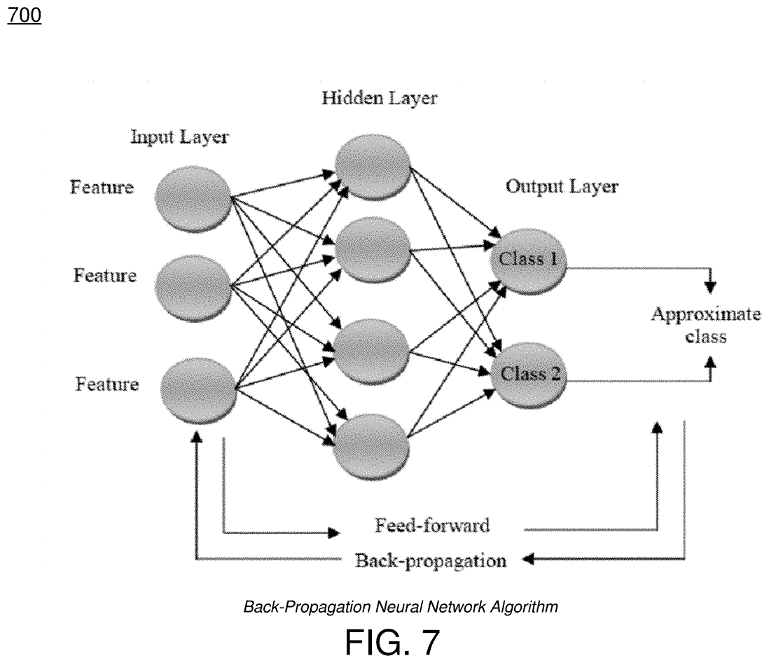

FIG. 7 illustrates an example of a deep learning neural network architecture used for storing and mapping relations of input-output model data. Deep learning [16-18] is one type of representational learning technique that can learn abstract mid-level and high-level features from image data. One advantage of deep learning is that it can learn extremely complex patterns. Deep learning algorithms, especially convolutional neural networks (CNNs), use “hidden layers” between inputs and outputs in order to model intermediary representations of image data that other algorithms cannot easily learn. Hence, they can generate high-level feature representations directly from raw medical images.

When dealing with ANNs, it is essential to utilize the advantages which represent human-brain-inspired architectures widely utilized in machine learning. ANN comprises three basic layers: the first is input one, the middle is hidden, and the final is output layer. The set of characteristics which represents the class that the ANN has to learn is received by the input layer. Further, input data processing is performed by the hidden layer through recognizing patterns to give identical or approximate value for the class that has to be recognized by the output layer. As depicted in FIG. 7, this process is expressed as feed-forward. If the output is not matched to the correct class, a back-propagation process is performed by the ANN for adjusting the connection weights of the corresponding hidden layers according to the calculated error, allowing correct class recognition based on repetitive learning iterations. This process of deep learning allows for less errors, better autonomic management of the patient data, diagnoses, and treatment offerings.

A recent alternative to ANN for big data, including images, is CNN. The basic difference between CNNs and ANNs is the convolution and pooling layers adopted in the former to extract image characteristics more effectively using fewer dimensions. A CNN passes a given image through its layers and outputs the decision class. The network may comprise tens or hundreds of layers, where each layer learns to detect different feature kinds. Each training image is subjected to filters at different resolutions, then the output of every convolved image is given as input to subsequent layer.

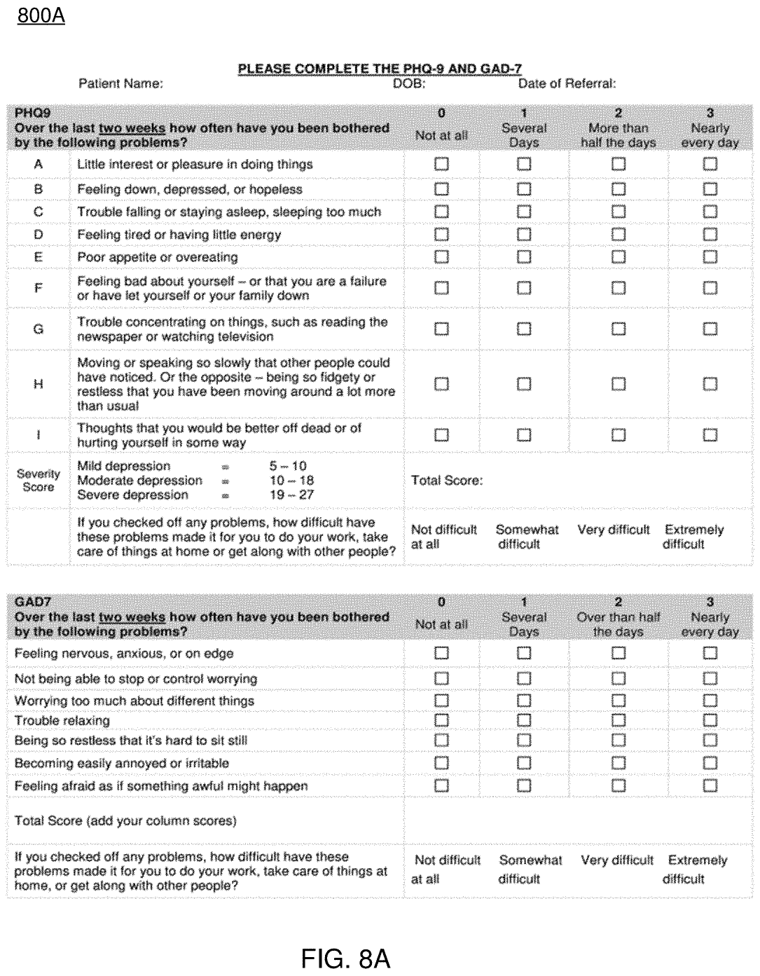

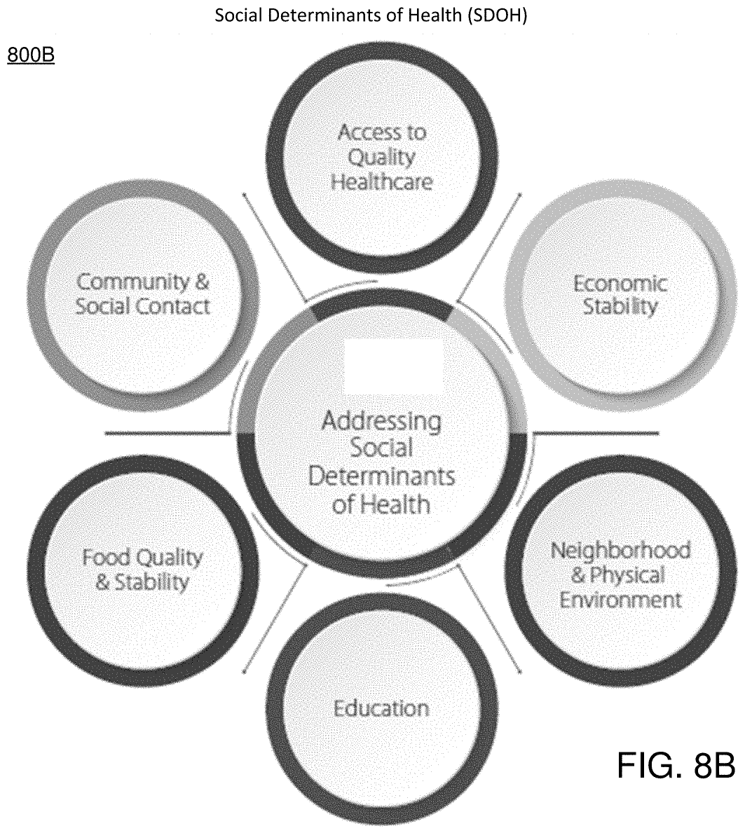

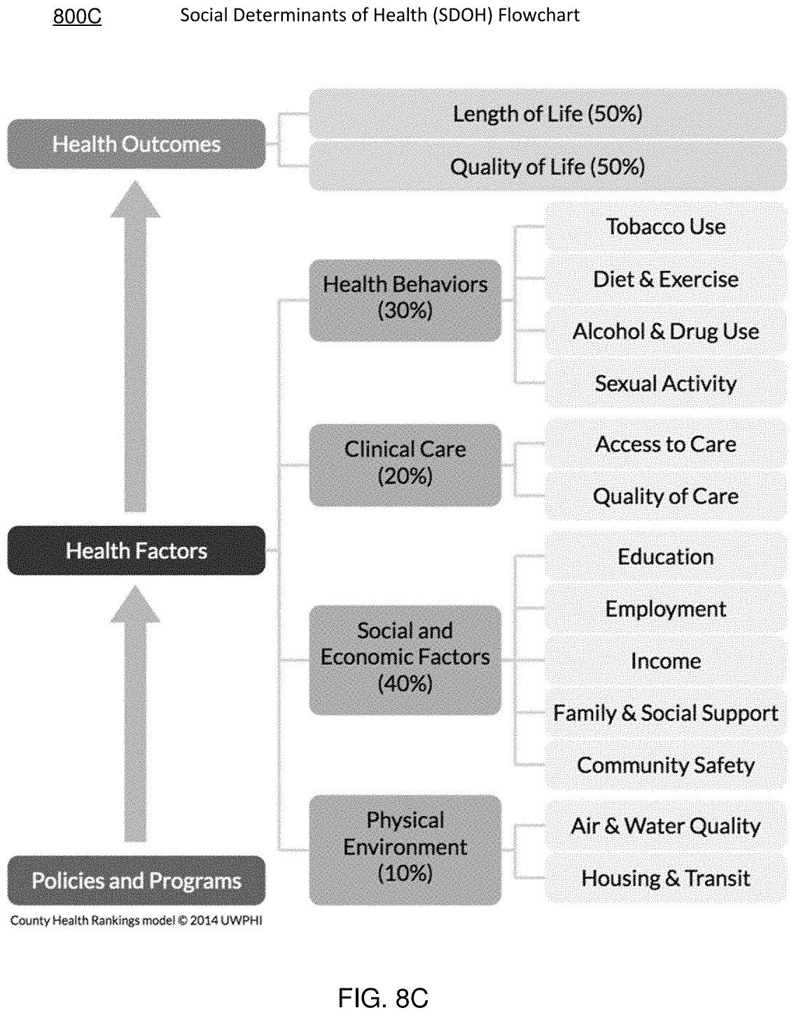

FIG. 8A, 800A illustrates assessment tools, Generalized Anxiety Disorder (GAD)-7 and Patient Health Questionnaire (PHQ)-9 screening tools, according to example embodiments. The Generalized Anxiety Disorder scale (GAD-7) is one of the most frequently used diagnostic self-report scales for screening, diagnosis, and severity assessment of anxiety disorder. Patient Health Questionnaire (PHQ)-9 is a self-administered version of the PRIME-MD diagnostic instrument for common mental disorders. If patients screen positive (>=10 for PHQ-9 and >=8 for GAD-7), then they may be referred to the Silver Cloud iCBT program via the eMedical Sentry platform, where the patients may have access to a full complement of remote patient monitoring (RPM) functionalities.

Other assessments and diagnostics tools may be used other than GAD-7 or PHQ-9 for predictive analytics or data analysis to improve remote patient monitoring and determining health outcomes. FIG. 8B, 800B illustrates Social Determinants of Health (SDOH) factors that can be used as data for assessment tools, datasets and predictive analytics utilized within the system including an AI module and a blockchain, according to example embodiments. Social Determinants of Health (SDOH) relates to using social determinants, the conditions in the places where people live, learn, work and play, as factors or determinants that affect a wide range of health risks and outcomes. This disclosure may utilize these SDOH patient metrics and other data including but not limited to available SDOH research data for predictive analytics about patient health outcomes. The present disclosure may utilize this information for preventing people from getting sick and optimizing positive outcomes when they are sick.

FIG. 8C, 800C illustrates a Social Determinants of Health (SDOH) Flowchart with factors that can be used as data for assessment tools, datasets and predictive analytics utilized within the system including an AI module and a blockchain, according to example embodiments. SDOH information may be used to in the development of more culturally competent assessments, better categorized data sets based SDOH information, more robust electronic health records, and patient information, and development of new treatments based on the intersection of various patient data points and key SDOH factors. The present disclosure may utilize these various developments to provide more effective, robust, and dynamic remote patient monitoring in real-time with actionable predictive analytics for patients, practitioners, and health service providers. Data and predictive analytics provided by the present disclosure may also prove useful in providing actionable data for local health administrators, lawmakers, and municipal governments for improving positive patient health outcomes, advance health equity, increase disease preventions, improve health education, and improve access to healthy foods.

In addition, a variety of predictive analytics can be run using various statistical models from data collected from IoT devices (e.g., wearable devices, electronic tattoos, medical diagnostic devices, MM, CT Scanners, Ultrasound, etc.). For example, while implantable cardiac sensors have been effective at reducing hospitalization for heart failure, wearable technology and noninvasive approaches including remote monitoring may be used with the predictive analytics system to predict heart failure rehospitalization. This system may utilize multi-sensor noninvasive remote monitoring for prediction of heart failure exacerbation. In one or more instances this novel remote monitoring system for diagnoses and predictive health outcomes can be used with various IoT devices (e.g., wearable devices, electronic tattoos, medical diagnostic devices, MM, CT Scanners, Ultrasound, etc.) to predict mortality, readmission, and/or emergency department visits. In one or more instances this novel remote monitoring system for diagnoses and predictive health outcomes can be used with various IoT devices (e.g., wearable devices, electronic tattoos, medical diagnostic devices, Mill, CT Scanners, Ultrasound, etc.) to improve quality of care, quality of life, minimize unnecessary invasive surgeries, prevent complications, aid in falls prevention, treat life-threatening situations, provide for urgent care interventions for certain categories of chronic patients. In one or more instances this novel remote monitoring system for diagnoses and predictive health outcomes can be used with various IoT devices (e.g., wearable devices, electronic tattoos, medical diagnostic devices, MRI, CT Scanners, Ultrasound, etc.) to improve the quality of home care, nursing care treatment, provide various means for quality control of health professionals, provide either real-time and/or continuous monitoring of patients with chronic conditions.

The above embodiments may be implemented in hardware, in a computer program executed by a processor, in firmware, or in a combination of the above. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

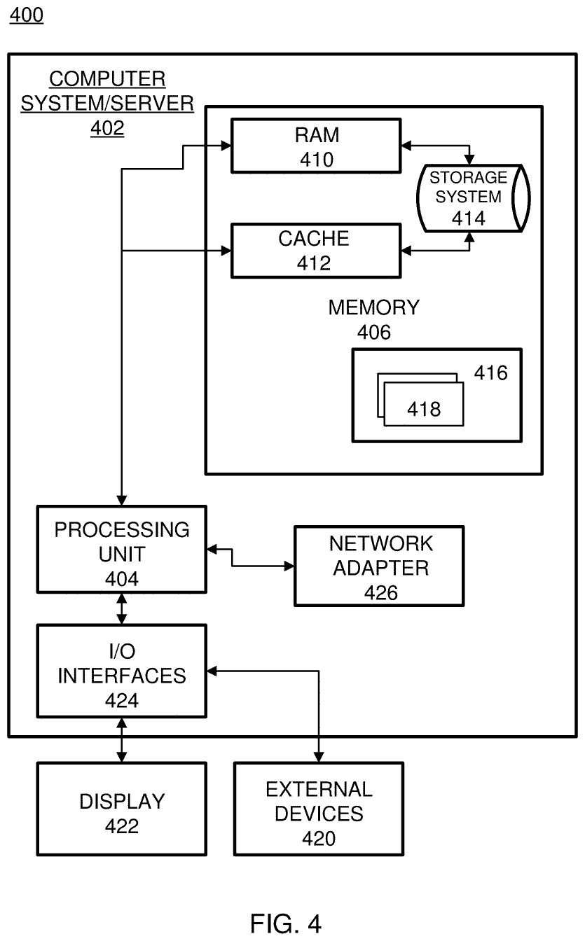

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 4 illustrates an example computer system/server node 400, which may represent or be integrated in any of the above-described components, etc.

FIG. 4 is not intended to suggest any limitation as to the scope of use or functionality of embodiments of the application described herein. Regardless, the computing node 400 is capable of being implemented and/or performing any of the functionality set forth hereinabove.

In the computing node 400 there is a computer system/server 402, which is operational with numerous other general purposes or special purpose computing system environments or configurations. Examples of well-known computing systems, environments, and/or configurations that may be suitable for use with computer system/server 402 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 402 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 402 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, the computer system/server 402 may be used in cloud computing node 400 shown in the form of a general-purpose computing device. The components of the computer system/server 402 may include, but are not limited to, one or more processors or processing units 404, a system memory 404, and a bus that couples various system components including system memory 404 to processor 404.

The bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The exemplary computer system/server 402 typically includes a variety of computer system readable media. Such media may be any available media that is accessible by the computer system/server 402, and it includes both volatile and non-volatile media, removable and non-removable media. System memory 404, in one embodiment, implements the flow diagrams of the other figures. The system memory 404 can include computer system readable media in the form of volatile memory, such as random-access memory (RAM) 410 and/or cache memory 412. The computer system/server 402 may further include other removable/non-removable, volatile/non-volatile computer system storage media. By way of example only, storage system 414 can be provided for reading from and writing to a non-removable, non-volatile magnetic media (not shown and typically called a “hard drive”). Although not shown, a magnetic disk drive for reading from and writing to a removable, non-volatile magnetic disk, and an optical disk drive for reading from or writing to a removable, non-volatile optical disk such as a CD-ROM, DVD-ROM or other optical media can be provided. In such instances, each can be connected to the bus by one or more data media interfaces. As will be further depicted and described below, memory 404 may include at least one program product having a set (e.g., at least one) of program modules that are configured to carry out the functions of various embodiments of the application.

Program/utility 414, having a set (at least one) of program modules 418, may be stored in memory 404 by way of example, and not limitation, as well as an operating system, one or more application programs, other program modules, and program data. Each of the operating system, one or more application programs, other program modules, and program data or some combination thereof, may include an implementation of a networking environment. Program modules 418 generally carry out the functions and/or methodologies of various embodiments of the application as described herein.

As will be appreciated by one skilled in the art, aspects of the present application may be embodied as a system, method, or computer program product. Accordingly, aspects of the present application may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present application may take the form of a computer program product embodied in one or more computer readable medium(s) having computer readable program code embodied thereon.

The computer system/server 402 may also communicate with one or more external devices 420 such as a keyboard, a pointing device, a display 422, etc.; one or more devices that enable a user to interact with computer system/server 402; and/or any devices (e.g., network card, modem, etc.) that enable computer system/server 402 to communicate with one or more other computing devices. Such communication can occur via I/O interfaces 424. Still yet, the computer system/server 402 can communicate with one or more networks such as a local area network (LAN), a general wide area network (WAN), and/or a public network (e.g., the Internet) via network adapter 424. As depicted, network adapter 424 communicates with the other components of computer system/server 402 via a bus. It should be understood that although not shown, other hardware and/or software components could be used in conjunction with computer system/server 402. Examples include, but are not limited to: microcode, device drivers, redundant processing units, external disk drive arrays, RAID systems, tape drives, and data archival storage systems, etc.

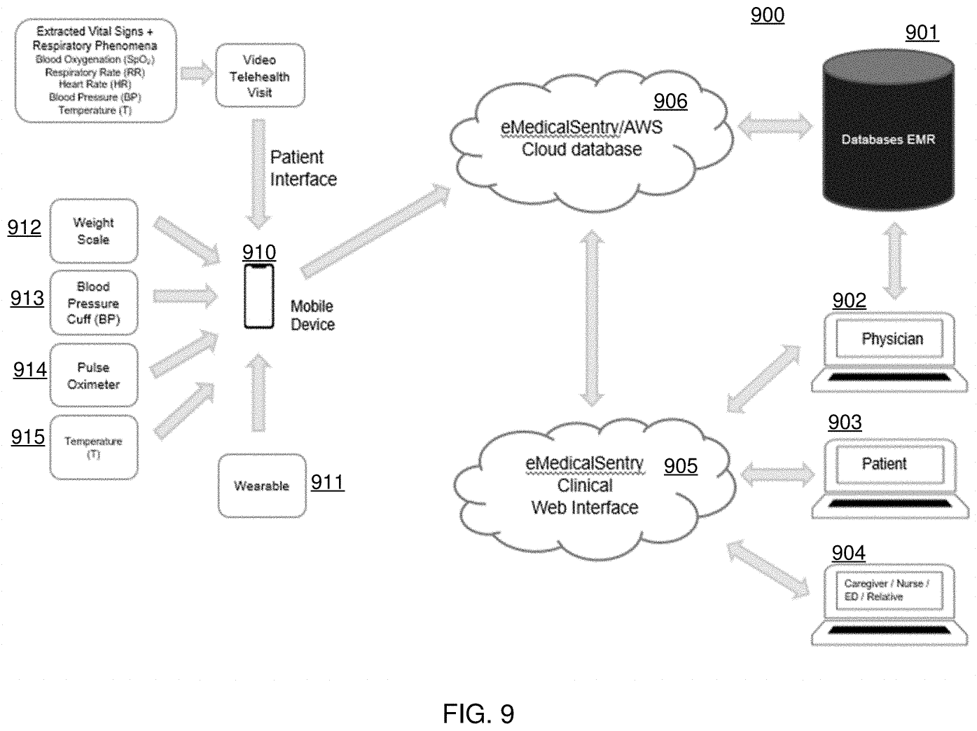

FIG. 9 illustrates an example of a system architecture for the eMedical Sentry system 900 that implements remote monitoring of a patient, and particularly supports the distinct video-based vital sign capture aspects disclosed herein. As described in detail herein, the eMedical Sentry system 900 can support enhanced remote patient monitoring using extracted vital signs from various forms of digital technology. In the example of FIG. 9, the system architecture of the eMedical Sentry system 900 includes: a mobile device 910, which is communicatively connected to several data collection devices shown as wearable device (e.g., smart watch) 911, weight scale 912, blood pressure cuff 913, pulse oximeter 914, and temperature device (e.g., electronic thermometer) 915; eMedical Sentry/AWS Cloud database network 906; an eMedical Sentry Clinical Web Interface 905; databases 901; a physician computer device 902; a patient computer device 903; and a medical personnel (e.g., caregiver, nurse, ED, relative) computer device 904. Vital signs that may be remotely obtained from a human patient utilizing the eMedical Sentry system 900 can include, but is not limited to: heart rate; blood pressure; oxygen saturation (e.g., SpO₂); body temperature; pulse rate; respiration rate; and other measurements of the body's functions that may requiring monitoring by medical professionals and health care providers. As will be described in greater detail herein, the eMedical Sentry system 900 enables a mobile device 910, which may be operated by the patient, as a type of optical sensor that can capture video images of the patient's body in order to ultimately obtain measurements for one or more vital signs of the patient. The mobile device 910, in accordance with the embodiment, can be implemented as any form of handheld and/or portable computer device that also has the capabilities to enable capturing video images, such as mobile telephone devices, smartphones, laptop computer devices, tablet computer devices, wearable devices, and the like. Imaging capabilities that are integrated and/or embedded in other types of devices other than mobile computer devices, such as modern security surveillance cameras, webcams, digital cameras, may also be employed to implement the disclosed video-based vital sign capture features of the eMedical Sentry system 900. Furthermore, the mobile device 910 can be configured to implement optical/image analysis techniques that can be employed to analyze the captured video image data in a manner that ultimately derives measurements of the patient's vital signs. As a general description, the mobile device 910 can be configured to implement color-based analysis techniques to obtain cardiac related parameters of vital signs from video image data of the patient's body, and to implement motion-based analysis techniques to obtain respiratory-related parameters of vital signs from video image data of the patient's body.

As illustrated in FIG. 9, the mobile device 910 can obtain various different physiological-related signals, also referred to as vital signs, from a patient either directly (e.g., video-based vital sign capture features) or from communication with one of the plurality of data collection devices 911-915 that may also interact directly with the patient. For example, a patient can interact with the weight scale 912, for instance standing on the device's 912 platform, which allows the weight scale 912 to measure the patient's weight. This measured data from the weight scale 912, which represents the patient's weight, can then be transmitted (e.g., wireless and/or wired data connection) to the mobile device 910. Continuing with this operation example, after the patient's weight is obtained measured by the weight scale 912 and collected by the mobile device 910, for instance as digital data which can represent and/or is related to a physiological vital sign for the patient, this data can be communicated to the eMedical Sentry/AWS Cloud database network 906. By communicating data from the mobile device 910 to the eMedical Sentry/AWS Cloud database network 906, this obtained data (e.g., representing with the patient's vital signs) can further be communicated to storage devices of the system 900, such as database 901, in order for the system 900 to persistently store and/or maintain vital sign data that corresponds to a particular patient.

Furthermore, FIG. 9 illustrates that the eMedical Sentry system 900 includes the eMedical Sentry Clinical Web Interface 905. As depicted in FIG. 9, eMedical Sentry Clinical Web Interface 905 is accessible to, and can be utilized by, a plurality of computer devices having access to the eMedical Sentry system 900, shown as computer devices 902-904. In an embodiment, the eMedical Sentry Clinical Web Interface 905 is an Internet-based platform that supports hardware and/or software components, such as software applications (apps) and interfaces (e.g., graphical user interfaces) that can be implemented on various types of computer devices, and which enable functions that allow data, communication, and interoperability to be supported amongst the distributed elements of the system 900. For example, data representing a patient's vital signs may be later accessed and/or retrieved by computer devices that can utilize such data (e.g., physician computer device 902, the patient computer device 903, and the medical personnel computer device 904) via GUIs that are implemented by the eMedical Sentry Clinical Web Interface 905. For example, the physician computer device 902 may have a GUI, implemented by the eMedical Sentry Clinical Web Interface 905, which allows a doctor to view data representing a patient's vital signs that are displayed in a visibly intelligible manner (e.g., graphs, charts, etc.), and which allows the data representing a patient's vital signs to be used in other medial related software on the computer device 902, such as Electronic Medical Records Software (EMR) or Electronic Health Records Software (EHR).

In an embodiment, the mobile device 910 is a mobile phone (e.g., smartphone) having an embedded digital camera therein, wherein the digital camera operates with high resolution (e.g., approximately 8 MP and higher) and within the visible light wavelength range (e.g., approximately 380-750 nm). The digital camera of the mobile device 910 can be used to implement a video camera imaging measuring technique for the system 900, also referred to as the video-based vital sign feature, in order to ultimately derive vital sign measurements of the patient. Accordingly, a mobile device 910 can be employed as a form of an optical sensor that records a video of the areas of the patient's body that are commonly associated with measuring, or otherwise assessing, vital signs such as the face, palm of the hand, and/or chest. Thus, in employing the video-based vital sign capture features of the eMedical Sentry system 900, the mobile device 910 can be used to capture video imaging data of different areas of the patient's body that are typically suitable for detecting/sensing vital signs (e.g., head, torso/chest, palm, etc.). The video imaging data that is captured by the mobile device 910 can then be subjected to an optical analysis process that obtains remote plethysmographic (rPPG) signals from the observed variations in light intensity within the imaging, also referred to herein as the color-based method. These rPPG signals are further analyzed to derive measurements for vital signs for the patient, such as blood pressure (BP) and heart rate (HR), which correspond to the areas of the patient's that were recorded by the mobile device 910. Therefore, the eMedical Sentry system 900 is capable of obtaining vital signs of the patient simply by capturing video recordings (e.g., capturing video imaging data) of the patient's body, which is a function on most mobile devices (e.g., tablet computers) and smartphones that is widely used in everyday life by many people. Consequently, even a lay person can use the system's 900 video-based vital sign capture features with a mobile device 910, and without physically touching the patient's body. In other words, the eMedical Sentry system 900 can utilize the mobile device 910 to obtain various vital signs of a patient, as oppossed to applying conventional vital sign measuring devices, where it may not be common knowledge to people outside of the medical industry how to properly use such devices and accurately obtain vital sign measurements. Moreover, these traditional vital sign measurement devices, for instance blood pressure cuffs, may be cumbersome and uncomfortable for the patient to use.

As an example, in the conventional method for obtaining a person's blood pressure as a vital sign, a patient would have to place a blood pressure cuff onto their own arm, which requires them to place and secure the instrument in the proper position on the arm in order to obtain an accurate reading for their blood pressure. In contrast, by employing the video-based vital sign capture features of the eMedical Sentry system 900 the patient only has to use the front-facing digital camera of their mobile device 910 (e.g., smartphone) as a type of optical sensor to take video(s) of areas of the body that may be related to measuring that particular vital sign, such as the forehead and the palm of the hand for blood pressure. Subsequently, other elements of the eMedical Sentry system 900, such as the physician computer device 902, may obtain the video imaging data obtained by the mobile device 910 via a communication network (e.g., Internet). These remote devices (with respect to the patient and/or the mobile device 910) of the system 900 can be equipped with the software and/or hardware functionality necessary to perform optical/image analysis techniques to the captured video imaging data, in accordance with the color-based analysis techniques described herein, in order to derive vital sign measurements from those video images of the patient's body. In this embodiment, the mobile device 910 does not need to perform the complex optical/image imaging analysis techniques associated with the system's 900 video-based vital sign capture features. Rather the mobile device 910 captures the video imaging data of the patient, while the optical/image analysis functions, such as color-based method and generating rPPG signals, are performed remotely by one or more other elements of the system 900. Furthermore, motion-based analysis, also referred to herein as motion-based method, can be utilized to perform optical/image analysis of video image data. Motion-based analysis is described in greater detail herein.

In another embodiment, the mobile device 910 can be configured with software and/or hardware functionality necessary to perform the optical/image analysis techniques necessary on the captured video imaging data, in accordance with the color-based analysis techniques described herein, in order to derive vital sign measurements. That is, the mobile device 910 can perform image analysis techniques in addition to, or in lieu of, the other elements of the system 900. In this embodiment, capturing video imaging data from the patient and analyzing the video imaging data is both performed on the mobile device 910, such that measurements of vital signs can be derived at the mobile device 910 communicated throughout the system 900, for instance to the physician computer device 902 and the medical personnel computer device 904, with requiring additional image analysis to be performed by the remote elements.

When visible light is used, and the bodily surface of a patient is recorded using video imaging capabilities, for instance using the digital camera of the mobile device 910, the pixels of each video's frame have an intensity level due to the light reflected by the body's surface over a two-dimensional grid of pixels. These intensity levels from the video image data of the patient's body can be analyzed in a manner that generates rPPG signals representing measured changes (in volume within an organ or portion of the body) in the patient's body, where these rPPG signals can be further analyzed to derive the measurements of the vital signs from the patient's body. In other words, the rPPG signals that are generated when the mobile device 910 is utilized as an optical sensor (i.e., video-based vital sign capture features of the system 900), is similar to the digital images that can be generated through other forms of optical sensors, like Charge Coupled Device (CCD) or Complementary Metal-Oxide Semiconductor (CMOS) sensors, which convert light radiations into electronic signals.

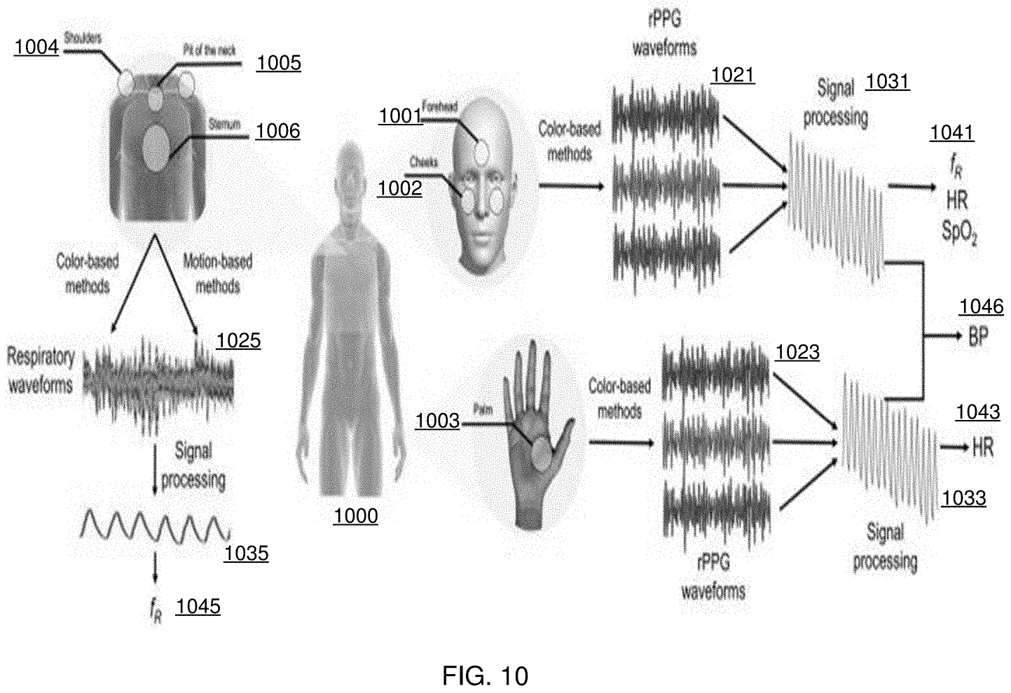

The mobile device 910 can be configured with optical/image analysis capabilities that enable the intensity changes of the pixels in the red color channel and in the blue color channel within the video image data to be considered. According to the embodiments, the video image data associated with the patient's body can be further analyzed and then used to employ a color-based technique that can detect changes in the patient's body to generate the aforementioned rPPG signals. For example, the color-based optical/image analysis can involve computing the ratio of the absorbances at the two wavelengths as they correspond to the red light (i.e., λ1=660 nm) and the IR light (i.e., λ2=940 nm), respectively, which can be used to generate the rPPG signal based on bodily changes, such as the cyclic movement of blood, that can be detected from the known absorbances. Examples of optical/image analysis processes that can be applied to video image data in order to implement the disclosed video-based vital sign capture features, specifically color-based analysis, and motion-based analysis, is depicted in FIG. 10, and thus is discussed in greater detail below in reference to FIG. 10.

Accordingly, the eMedical Sentry system 900, as disclosed herein, implements a novel approach to remote medical monitoring of a patient's vital signs by employing the system's 900 video-based vital sign capture features, which provides several improved and enhanced capabilities over commonly used video-based remote patient medical screening/doctor visit methods. For example, the system's 900 based vital sign capture features can be used as a distinct and improved alternative to commonly used cardiovascular disease detection techniques, by enabling early disease detection via initial presentation (e.g., remotely capturing the patient's vital signs associated with cardiac-related parameters) and prevention through improved management of chronic asthma symptoms. This innovative technology of the system 900, which enables a mobile device to serve as an optical sensor to remotely monitor a patient (e.g., using video-based vital sign capture), has the capability to: (i) extend the reach for physicians and accessibility for patients; (ii) improve efficiency of healthcare delivery to the highest risk patients; (iii) limit exposure to infectious disease for both patient and provider; and (iv) utilize common (relatively) commercial off the shelf (COTS) technologies instead of expensive boutique equipment.

FIG. 10 illustrates an example of optical/image analysis techniques that can be applied to video data of a patient's 1000 body in order to implement the aforementioned video-based vital sign capture features, as disclosed herein. In particular, FIG. 10 depicts that video image data from several areas of the patient's 1000 body can be captured and analyzed in a manner that generates physiological-related signals for the patient, such rPPG signals that represent the monitoring of the patient's biological functions like blood pressure, heart rate, and the like. As previously described, a video-enabled imaging device, such as a digital camera embedded in a smartphone, can function as an optical sensor that has the capability to work in a visible light wavelength range (approximately 380-750 nm) and can retrieve physiological-related signals and/or vital signs, from different body areas of the patient 1000. Particularly, FIG. 10 depicts an example of capturing video image data (e.g., from creating a video recording using the digital camera of a smartphone) corresponding to multiple areas of the patient's 1000 body, including: the forehead 1001; cheeks 1002; palm 1003; shoulders 1004; pit of the neck 10005; and sternum 1006.

Subsequently, the video image data corresponding to these areas 10001-1006 of the patient's 1000 body are subjected to optical/image analysis, wherein the optical/image analysis can involve color-based analysis techniques and/or motion-based analysis techniques, which generate signals and/or waveforms that represent measurable changes in the patient's body, such as rPPG signals. Consequently, both color-based analysis techniques and/or motion-based analysis techniques can generate signals from video image data of the patient 1000 that are ultimately used to measure vital signs for the patient.

Color-based analysis, also referred to herein as the color-based method, relies on the detection of subtle skin color changes of the patient 1000 that can be captured in video images of the patient's 1000 body, due to the cyclical movement of the patient's 1000 blood. These color changes can be detected by analyzing any changes/variations of light intensity in pixels of the captured video image, as will be described in greater detail below. These detected color-based changes in the patient's 10000 body (from analyzing video image data) can be represented by plethysmographic (i.e., PPG) signals, which is typically referred to as remote-PPG (sometimes reported as rPPG) or imaging-PPG (i-PPG in short). As a general description, generating rPPG signals is based on the principle that blood absorbs light than the surrounding tissue, thus blood volume variations affect light transmission and reflectance, for instance causing changes in light intensity when less blood is present (thereby absorbing less light) or when more blood is present, (thereby absorbing more light). As FIG. 10 illustrates, the measurements (e.g., estimations) of cardiac-related parameters of the patient's 1000 vital signs, such as vital signs (like heart rate) that may be obtained from body areas 1001-1003 of the patient's 1000 body (which are commonly monitored to assess the patient's heart and/or cardiovascular function), are obtained by applying color-based analysis to the video image data captured of those body areas 1001-1003, where the color-based analysis generates rPPG signals that represent biological changes (e.g., circulation of blood) in those corresponding body areas 1001-1003. For example, color-based analysis to generate the rPPG signals 1021, 1023 which correspond to the patient's body areas 1001-1003 can involve analyzing the intensity changes of the pixels of the video image data specifically in the green color channel since the hemoglobin has a high ability of absorption in this color channel.

In the example of FIG. 10, color-based methods are utilized to generate rPPG waveforms 1021 from the images captured of the patient's 1000 facial area, namely the forehead 1001 and cheeks 1002. Further, FIG. 10 illustrates that applying a signal processing technique 1031, which can be performed remotely form the patient 1000 in accordance with the disclosed embodiments, can be used to analyze the resulting rPPG waveforms 1021 converting the light intensities represented in the signal into digital data that can be used to derive vita sign data associated with the patient, such as fR , HR, and SpO₂ information 1041. It is noted that when HR is estimated from rPPG, for instance HR information 1041 estimated from rPPG waveforms 1021, it is typically termed as pulse rate. Similarly, color-based methods are utilized to generate rPPG waveforms 1023 from the images captured of the patient's 1000 hand area, namely the palm 1003. Here, applying a signal processing technique 1033 can be used to analyze the resulting rPPG waveforms 1023 in order to derive HR information 1043. FIG. 10 also illustrates that data from signal processing 1031 and signal processing 1033 can be combined in a manner that derives BP information 1046 for the patient 1000. In addition, color-based methods and motion-based methods are depicted in FIG. 10 to generate respiratory waveforms 1025 from the images captured of the patient's 1000 chest area, namely the shoulders 1004, neck 1005, sternum 1006. Here, applying a signal processing technique 1035 can be used to analyze the resulting respiratory waveforms 1025 in order to derive fR information 1045.

The measuring of cardiac-related parameters of the patient's 1000 vital signs depends on the acquisition of a rPPG signal, such as rPPG waveforms 1021, 1023, and 1025, which is obtained by analyzing the intensity changes of the pixels in the green color channel since the hemoglobin in blood has high ability of absorption in this channel. A measurement of SpO₂, such as the SpO₂ information 1041 derived from rPPG waveform 1021, requires light at two different wavelengths, which is consistent with the depiction of the rPPG waveform 1021, for example, being a composite of light in the green color wavelength, red color length, and blue color length. SpO₂ is defined as the ratio between the oxygenated hemoglobin (HbO₂) and the total amount of hemoglobin (i.e., deoxygenated, and oxygenated, hemoglobin), which can be optically distinguished by the different absorption of light at two different wavelengths (i.e., oxyhemoglobin has high absorption at infrared light (IR) and deoxyhemoglobin has a higher absorption at red light).

Further, in the example of FIG. 10, the optic/image analysis of the imaging data captured of the patient's 1000 chest/torso area, namely the shoulders 1004, neck 1005, and sternum 1006, involves color-based analysis and/or motion-based analysis to generate respiratory waveforms 1025 from these images. Motion-based analysis, as disclosed herein, is an image analysis technique that is based on the detection of small-amplitude movements recorded by a digital camera (e.g., video camera imaging functions) of a mobile device (as described in above in reference to FIG. 9). In this example, utilizing a mobile device as an optical sensor for monitoring respiratory-related parameters of the patient's 1000 vital signs, relies on the detection of the thorax movements caused by the patient's 1000 breathing activity captured by the video imaging data. Movement of the recorded surfaces (i.e., chest wall movements due to the respiratory activity) of the patient's 1000 body affects the changes of intensity of the pixels, the resulting changes of the reflected light intensity may be used to collect respiratory-related motion, such as breathing patterns and other related respiratory parameters indirectly. Additionally, motion-based analysis that is used to analyze the video imaging data captured from the patient's 1000 shoulders 1004, neck 1005, and sternum 1006 can involve another approach that is related to the detection of optical flow, which can be used to detect the chest surface movement of the patient 1000. Optical flow enables computing the displacement between two consecutive frames of video images (of the video imaging data) by tracking the features of the images, where calculating displacement of chest motion enables of form of measurement of respiratory parameters of the patient's 1000 vital signs. Motion-based analysis can also be used to measure the aforementioned cardiac-related parameters of the patient's vital signs. In this case, motion-based analysis is based on the detection of the head movement due to the movement of the blood from the heart to the head, which is sometimes known as ballistocardiography (BCG). Feature tracking can be used to extract the motion of the patient's 1000 head. In some embodiments, motion-based analysis involves tracking/extracting motion related to the patient's 1000 facial/head area in the vertical direction, where the vertical direction may be the most optimal axis to measure the upright movement of the head.

Referring back to FIG. 10, by utilizing the abovementioned approaches for motion-based analysis, such as the light intensity approach and the optical flow approach (e.g., feature tracking/extraction), the respiratory waveforms 1025 which represents respiratory-related parameters of the patient's 1000 vital signs can be generated from performing this form of optical/image analysis from the captured video imaging data of the patient 100. Accordingly, these optic/image analysis techniques depicted in FIG. 10 accomplishes an enhanced remote patient monitoring that is capable of ascertaining both respiratory-related parameters and cardiac-related parameters of the patient's vital signs.

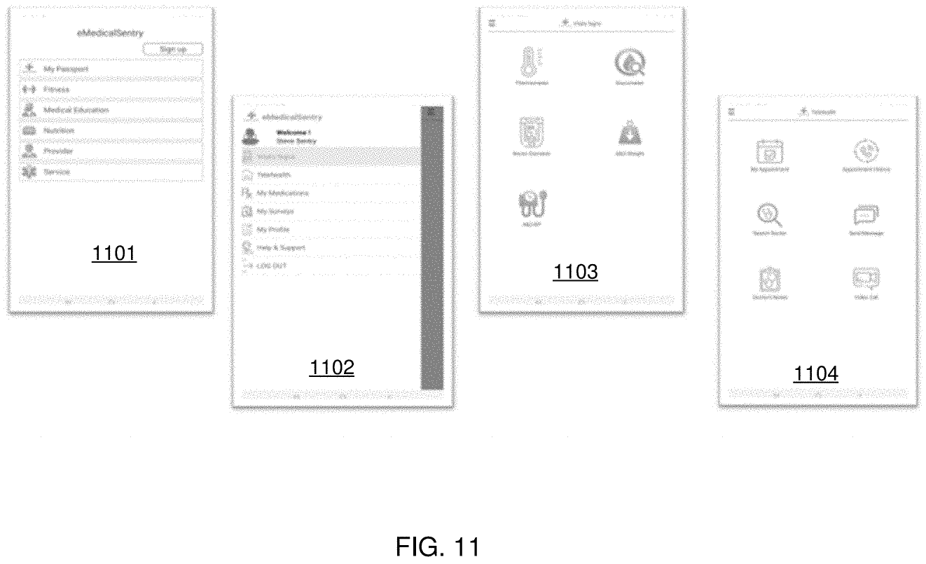

Referring now to FIG. 11, examples of various GUIs 1101-1104 that may be generated by an eMedical Sentry (eMS) patient software application (app) associated with the eMedical Sentry system, such as the eMedical Sentry Clinical Web Interface (shown in FIG. 9) for interactive use by an end user, such as a medical professional (e.g., doctor, nurse, etc.) are depicted. According to the embodiments, the GUIs 1101-1104 are particularly illustrated in an ordered sequence that may be encountered by the user as they navigate (e.g., scroll) through the various options supported by the eMS patient app. As seen in FIG. 11, this sequence can include: an eMedicalSentry System home GUI 1101; a log-in user “welcome” GUI 1102; a vital signs GUI 1103; and a telehealth GUI 1104. Accordingly, an end user can interact with GUIs 1101-1104 to further access the various other GUIs and functions of the system that are made available via the eMS patient app. In an embodiment, the patient software app, and the associated GUIs 1101-1104 may be displayed on a client device, such as smartphone or tablet computer device.

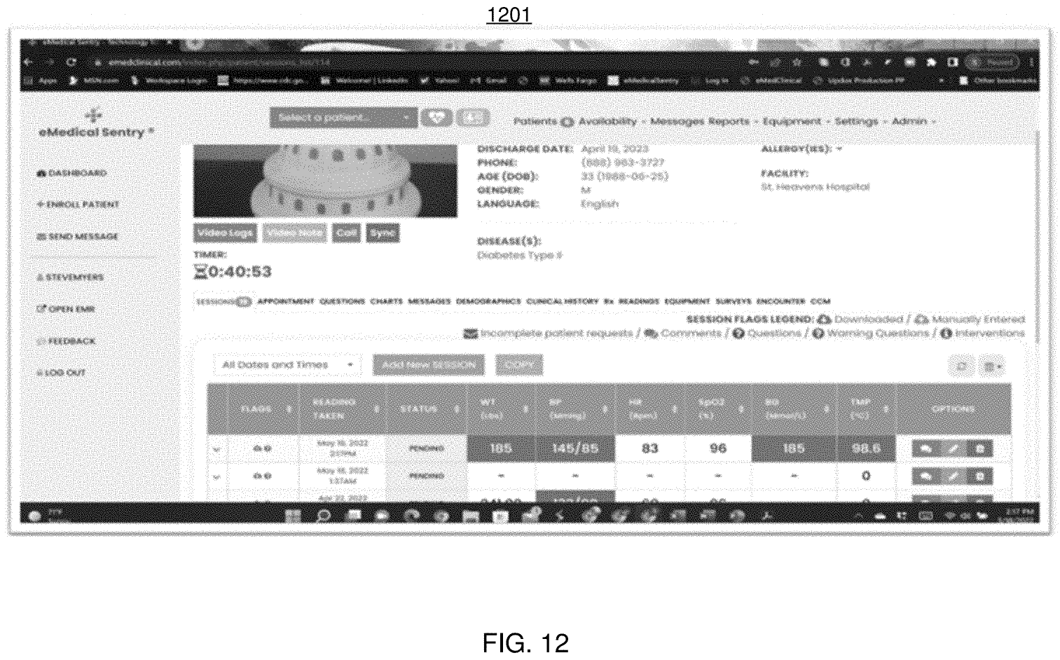

Referring now to FIG. 12, an example of a GUI 1201 that may be generated by a web-based software application (app) associated with the eMedical Sentry system, such as the eMedical Sentry Clinical Web Interface (shown in FIG. 9) for interactive use by an end user, such as a medical professional (e.g., doctor, nurse, etc.) is depicted. According to the embodiments, the GUI 1201 generated by the eMedical Sentry (eMS) Clinical Web app includes interactive selections such as “dashboard”, “enroll patient”, and “send message”. Accordingly, an end user can interact with GUI 1201 to further access the various other GUIs and functions of the system that are made available via the eMS Clinical Web app. In an embodiment, the eMedical Sentry Clinical Web app, and the associated GUI 1201 may be displayed on a client computer device, such as smartphone, laptop computer, desktop computer, or tablet computer device.

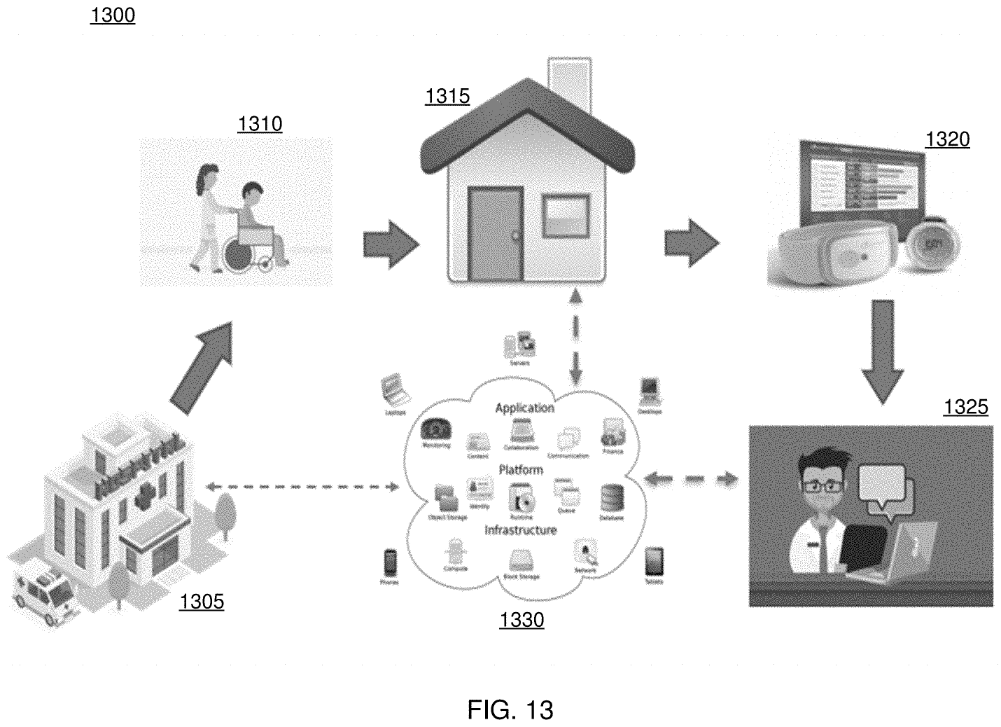

FIG. 13 illustrates an example deployment scenario 1300 of various elements of the eMedical Sentry system, as disclosed herein. FIG. 13 depicts a hospital 1305 where a patient may be physically present, for instance receiving medical care or for a regularly scheduled check-up. Next, FIG. 13 illustrates the patient being discharged 1310 from the hospital, which allows them to return to their home 1315. At their home 1315, which is considered remote with respect to the hospital 1305, the patient can personally utilize several patient devices 1320 that are communicatively connected to the eMedical Sentry system 1330. The patient devices 1320 and the eMedical Sentry system 1330 are connected in a manner that allows these patient devices 1320 to be employed by the patient to access and/or utilize the various functions for enhanced remote monitoring for the patient that are supported by the system. For example, the patient can access a software application of the system's platform using their personal laptop, use a blood pressure cuff, and a wearable device (e.g., smartwatch) as the patient devices 1320 in order to capture information relating to measuring the patient's vital signs at their home 1315, which is communicated to the system 1330. As a key feature of the system 1330, the patient devices 1320 can include a smartphone that can be used to implement the video-based vital sign capture features, as disclosed herein. The patient devices 1320 can be any of the collection devices and the mobile device (operating as an optical sensor) that are previously described in reference to FIG. 9.

The system 1330 is shown to include various software applications, the platform, and infrastructure that is necessary to process this information that is captured by the patient's devices 1320 and communicated from the patient's home 1315, which enables the system to remotely monitor the patient's vital signs, even after they have left the hospital 1305. The system 1330 can include any of the aforementioned elements of the eMedical Sentry system that are described in reference to FIG. 9.

Additionally, FIG. 13 depicts that a computer device 1325 associated with a medical professional, such as a doctor, is also communicatively connected to the system 1330 and the patient devices 1320 in a manner that allows information relating to the patient's vital signs that has been remotely captured at the patient's home to be accessed and/or visible to the medical professional via their computer device 1325. Thus, the medical professional can be directly involved in the remote monitoring process for the patient, as the medical professional can access the patient's information, such as their vital signs, from the system 1330 to become aware of any medical issues that the patient may be experiencing even when that medical professional is not physically proximate to the patient.

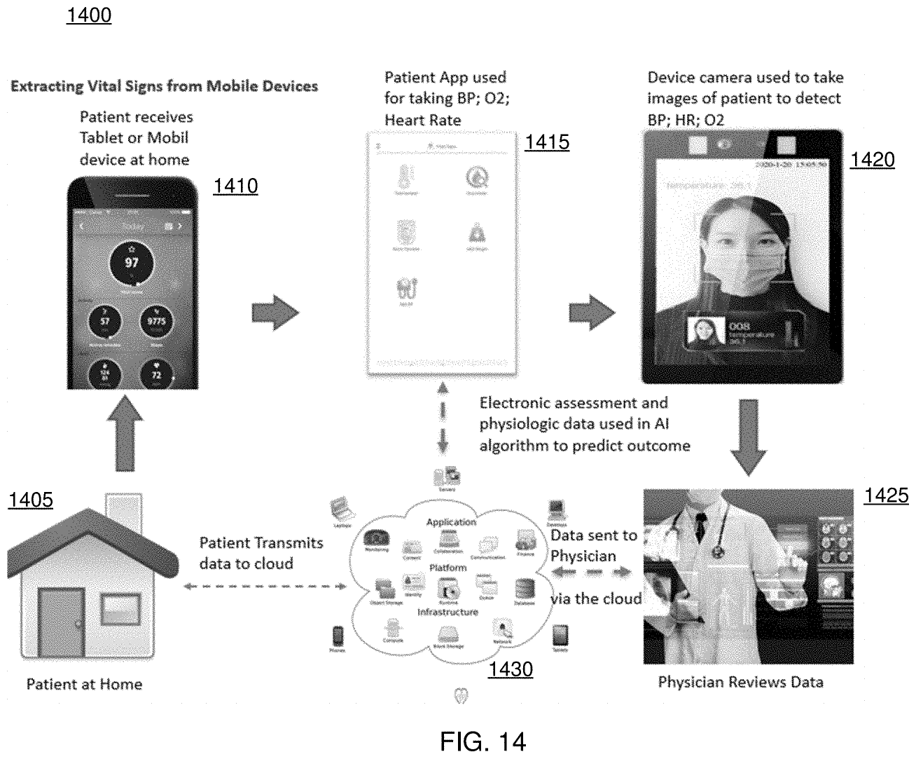

FIG. 14 depicts an operational example of remotely monitoring a patient's vital signs by employing a mobile device, specifically in accordance with the video-based vital sign capture features, as disclosed herein. FIG. 14 illustrates that at a patient's home 1405, the patient can receive a mobile device 1410 that is associated with the system. The mobile device 1410 can be implemented as a tablet computer or a smartphone device, for example. The patient can then access a patient app 1415 that is implemented by the system, via their mobile device 1410. The patient can interact with GUIs of the patient app 1415 that are displayed on their mobile device 1410, which allows the user to access and/or utilize various features of the system, and particularly the patient can select an option on the patient app 1415 that initiates video-based vital sign capture feature. FIG. 14 illustrates that the patient app 1415 can be selected for initiating the remote monitoring of several vital signs for the patient, namely blood pressure (BP), oxygen levels (02), and heart rate (HR), using the mobile device 1410. The patient app 1415 can be implemented as the eMS patient app that is described in greater detail in reference to FIG. 11.

Further, FIG. 14 depicts that a camera 1420 of the mobile device 1410 is employed to take video images of the patient in order to detect, or otherwise measure, the patient's vital signs. For example, the camera 1420 can be a digital camera that is embedded in a tablet computer and has high resolution capabilities. As seen, a particular area of the patient's body, which is the head/facial area of the patient in FIG. 14, is recorded by the camera 1420 to capture video imaging data of the patient that can be further analyzed in order to measure their associated vital signs. According to the embodiments, the video imaging data of the patient that is captured by the camera 1420 is processed using the optical/image analysis techniques, such as motion-based analysis and color-based analysis, which are described in greater detail in reference to FIG. 10 in a manner that derives measurements for the patient's vital signs, including blood pressure, heart rate, and oxygen levels. Subsequently, these measurements of the patient's vital signs, which are captured by employing the mobile device 1410 as an optical sensor, are communicated to a medical professional, shown as physician 1425 for their review. Consequently, the physician 1425 can monitor the vital signs of the patient remotely via the mobile device 1410, which allows the physician to detect signs of chronic illness in the patient (based on their measured vital signs), such as asthma, early and without having to be physically present in the same vicinity as the patient.

FIG. 14 also illustrates that the system 1430 can receive patient data (e.g., from the patient) and/or transmit patient data (e.g., to the physician) over a communication network, such as the Internet. The system 1430 can also predict outcomes relating to the patient health, by analyzing electronic assessment and physiological data using AI technology.

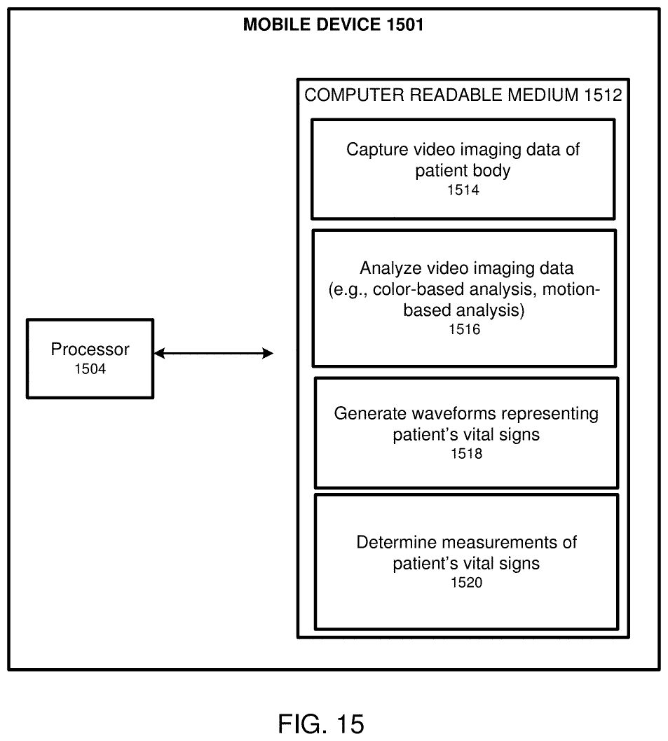

FIG. 15 illustrates an example computer system, depicted as a mobile device 1501 that is configured to execute the video-based vital sign capture capabilities, in accordance with embodiments of the present invention.

The mobile device 1501 may be a computing device, such as smartphone or a laptop computer, or the like, and may include a processor 1504, which may be a semiconductor-based microprocessor, a central processing unit (CPU), an application specific integrated circuit (ASIC), a field-programmable gate array (FPGA), and/or another hardware device. Although a single processor 1504 is depicted, it should be understood that the mobile device 1501 may include multiple processors, multiple cores, or the like, without departing from the scope of the invention. In the example, processor 1504, that can be implemented as a hardware, such as a semiconductor-based microprocessor, a central processing unit (CPU), an application specific integrated circuit (ASIC), a field-programmable gate array (FPGA), and/or another hardware device, software, firmware, or any combination thereof on the mobile device 1501.

The mobile device 1501 may also include a non-transitory computer readable medium 1512 that may have stored thereon machine-readable instructions executable by the processor 1504. Examples of the machine-readable instructions are shown as 1514-1520 and are further discussed below. Examples of the non-transitory computer readable medium 1512 may include an electronic, magnetic, optical, or other physical storage device that contains or stores executable instructions. For example, the non-transitory computer readable medium 1512 may be a Random-Access memory (RAM), an Electrically Erasable Programmable Read-Only Memory (EEPROM), a hard disk, an optical disc, or other type of storage device.

The processor 1504 may fetch, decode, and execute the machine-readable instructions 1514 to capture video imaging data of the patient's body. A video imaging device, such as a digital camera embedded in the mobile device 1501 may be utilized to capture video images of the patient's body that are related to physiological functions of the patient, or vital signs. Examples of vital signs that may be measured using the mobile device 1501 can include heart rate, blood pressure, oxygen levels, and the like. According to the embodiments, the video imaging device used to the capture the video imaging data of the patient operates in the visible light wavelength range (e.g., 380-750 nm) and functions with high resolution. In the embodiments, the captured video imaging data is obtained from recording particular areas of the patient's body that are related to measuring physiological functions and/or vital signs, such as head/facial area (e.g., forehead, cheeks, etc.), hand area (e.g., palms, wrists, etc.), and chest area (e.g., shoulders, sternum, etc.), and the like.

The processor 1504 may fetch, decode, and execute the machine-readable instructions 1516 to analyze the video imaging data using optic/image analysis techniques described herein. According to the embodiments, analyzing the video imaging data can include applying the color-based analysis techniques and/or the motion-based analysis techniques as described in detail in reference to FIG. 10. Color-based analysis techniques can be used for monitoring of cardiac-related parameters of the patient's vital signs. Motion-based techniques can be used for monitoring of respiratory-related parameters of the patient's vital signs.

The processor 1504 may fetch, decode, and execute the machine-readable instructions to generate waveforms that represent that physiological changes in the patient's body that can be used to derived measurements of the vital sign. These waveforms can include rPPG signals and/or respiratory signals. According to the embodiments, these waveforms are generated using the process and/or techniques described in detail in reference to FIG. 10. The waveforms represent changes in the patient's physiological functions, such as variation in blood volume that can be used to derive heart rate, blood pressure, and oxygen levels as vital signs measurements associated with the patient.

The processor 1504 may fetch, decode, and execute the machine-readable instructions 1520 to determine measurements of the patient's vital signs from the generated waveforms. Accordingly, the processor 1504 executes a video-based measurement of the patient's vital signs, which can further be utilized for enhanced remote monitoring of the patient's health, including their vital signs, via the mobile device 1501.

The above embodiments may be implemented in hardware, in a computer program executed by a processor, in firmware, or in a combination of the above. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

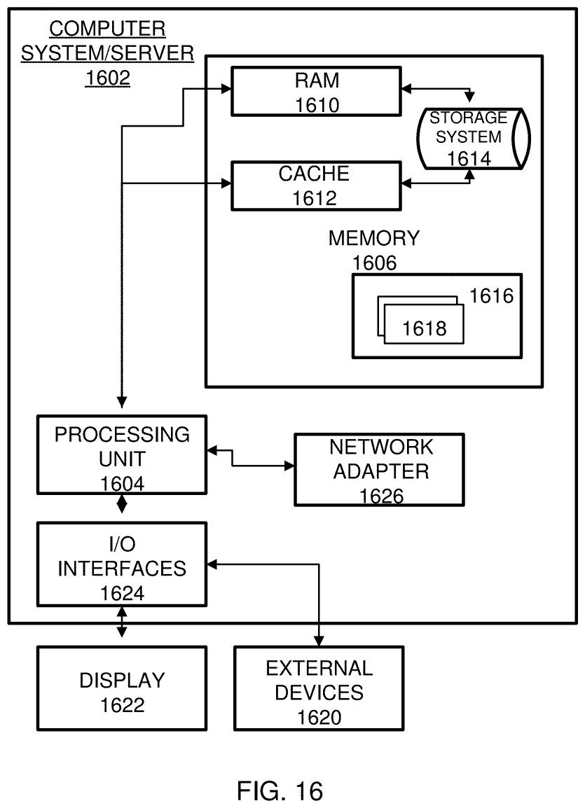

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 16 illustrates an example computer system 1600, for example a mobile device that implements the video-based vital sign capture capabilities, as disclosed herein.

FIG. 16 is not intended to suggest any limitation as to the scope of use or functionality of embodiments of the application described herein. Regardless, the computing node 1600 is capable of being implemented and/or performing any of the functionality set forth hereinabove .

In the computing node 1600 there is a computer system 1602, which is operational with numerous other general purposes or special purpose computing system environments or configurations. Examples of well-known computing systems, environments, and/or configurations that may be suitable for use with computer system 1602 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 1602 may be described in the general context of computer system-executable instructions, such as program modules, being executed by a computer system. Generally, program modules may include routines, programs, objects, components, logic, data structures, and so on that perform particular tasks or implement particular abstract data types. Computer system 1602 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 16, the computer system 1602 may be used in cloud computing node 900 shown in the form of a general-purpose computing device. The components of the computer system 1602 may include, but are not limited to, one or more processors or processing units 1604, a system memory 1606, and a bus that couples various system components including system memory 1606 to processor 1604.

The bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The exemplary computer system 1602 typically includes a variety of computer system readable media. Such media may be any available media that is accessible by the computer system 1602, and it includes both volatile and non-volatile media, removable and non-removable media. System memory 1606, in one embodiment, implements the flow diagrams of the other figures. The system memory 1606 can include computer system readable media in the form of volatile memory, such as random-access memory (RAM) 1610 and/or cache memory 1612. The computer system 1602 may further include other removable/non-removable, volatile/non-volatile computer system storage media. By way of example only, storage system 514 can be provided for reading from and writing to a non-removable, non-volatile magnetic media (not shown and typically called a “hard drive”). Although not shown, a magnetic disk drive for reading from and writing to a removable, non-volatile magnetic disk, and an optical disk drive for reading from or writing to a removable, non-volatile optical disk such as a CD-ROM, DVD-ROM or other optical media can be provided. In such instances, each can be connected to the bus by one or more data media interfaces. As will be further depicted and described below, memory 1606 may include at least one program product having a set (e.g., at least one) of program modules that are configured to carry out the functions of various embodiments of the application.

Program/utility 1616, having a set (at least one) of program modules 1618, may be stored in memory 1606 by way of example, and not limitation, as well as an operating system, one or more application programs, other program modules, and program data. Each of the operating system, one or more application programs, other program modules, and program data or some combination thereof, may include an implementation of a networking environment. Program modules 518 generally carry out the functions and/or methodologies of various embodiments of the application as described herein.

As will be appreciated by one skilled in the art, aspects of the present application may be embodied as a system, method, or computer program product. Accordingly, aspects of the present application may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present application may take the form of a computer program product embodied in one or more computer readable medium(s) having computer readable program code embodied thereon.

The computer system 1602 may also communicate with one or more external devices 1620 such as a keyboard, a pointing device, a display 1622, etc.; one or more devices that enable a user to interact with computer system 1602; and/or any devices (e.g., network card, modem, etc.) that enable computer system 1602 to communicate with one or more other computing devices. Such communication can occur via I/O interfaces 1624. Still yet, the computer system 1602 can communicate with one or more networks such as a local area network (LAN), a general wide area network (WAN), and/or a public network (e.g., the Internet) via network adapter 1626. As depicted, network adapter 1626 communicates with the other components of computer system 1602 via a bus. It should be understood that although not shown, other hardware and/or software components could be used in conjunction with computer system 1602. Examples include, but are not limited to: microcode, device drivers, redundant processing units, external disk drive arrays, RAID systems, tape drives, and data archival storage systems, etc.

Although an exemplary embodiment of at least one of a system, method, and non-transitory computer readable medium has been illustrated in the accompanied drawings and described in the foregoing detailed description, it will be understood that the application is not limited to the embodiments disclosed, but is capable of numerous rearrangements, modifications, and substitutions as set forth and defined by the following claims. For example, the capabilities of the system of the various figures can be performed by one or more of the modules or components described herein or in a distributed architecture and may include a transmitter, recipient or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a Smart phone or any other suitable computing device, or combination of devices. Presenting the above-described functions as being performed by a “system” is not intended to limit the scope of the present application in any way but is intended to provide one example of many embodiments. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed but is merely representative of selected embodiments of the application.

One having ordinary skill in the art will readily understand that the above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms, etc.) thereto.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A system, comprising:
an Internet-based cognitive behavioral therapy (iCBT) system collecting patient data associated with assessment and screening of a patient;
a remote patient monitoring system collecting patient data associated with remote and real-time monitoring of the patient;
an artificial intelligent (AI) machine learning system collecting patient data associated with health diagnosis and predictions for the patient; and
an integrated system receiving the patient from the iCBT system, the patient data from the remote patient monitoring system, and the patient data from the AI machine learning system, and analyzing the combination of the patient data to enable evidence-based interventions, diagnoses, and predictive health for the patient.

22. The system of claim 21, wherein the remote patient monitoring system comprises:
a plurality of communication points comprising one or more of: an emergency response service, biometric monitoring; video communication, interactive voice response (IVR), medical monitoring, clinical telecare, and Internet of Things (IoT) devices.

23. The system of claim 21, wherein the patient is non-white and is associated with cardiovascular disease (CVD) and depression/anxiety.

24. The system of claim 23, wherein the patient data associated with assessment and screening of the patient comprises digital screening of the patient for depression/anxiety using Generalized Anxiety Disorder 7 (GAD-7) and Patient Health Questionnaire 9 (PHQ-9) screening tools.

25. The system of claim 24, wherein the iCBT system refers the patient to a full complement of functionalities of the remote patient monitoring system upon screening positive for depression/anxiety based on the GAD-7 and PHQ-9 screening tools.

26. The system of claim 25, wherein the functionalities of the remote patient monitoring system comprise: video-based vital sign capture; diagnosis of health associated with the patient predictive health outcomes associated with the patient and employing one or more IoT devices.

27. The system of claim 26, wherein employing the one or more IoT devices enables additional functionalities of the remote patient monitoring system, the additional functionalities comprising:
improving quality of care;
minimization of invasive surgeries;
complication prevention;
fall prevention;
treating life-threatening situation prevention;
urgent care interventions;
improving quality of home care;
improving nursing care treatment
quality control of health professionals;
real-time monitoring of chronic conditions associated with the patient and
continuous monitoring of chronic conditions associated with the patient.

28. The system of claim 21, wherein the integrated system generates treatment and diagnoses of disease and maladies of the patient using remote monitoring, data analytics, and therapies.

29. The system of claim 28, wherein the data analytics is associated with various populations comprising one or more of: non-white populations; minority populations; and
underserved populations.

30. The system of claim 21, further comprising:
a diagnostic server connected to the AI machine learning system via a network, the diagnostic server collecting data from one or more medical data silos; and
remote users connected to the diagnostic server via the network, wherein the remote users comprise doctors and patients, wherein the AI machine learning system receives the collected data as training data, trains machine learning models using the training data to generate the patient data associated with health diagnosis and predictions for the patient, and provides AI assisted remote patient monitoring, data collection, and data analysis.

31. The system of claim 30, wherein the diagnostic server collects training data from a ledger of a blockchain to train the machine learning models.

32. The system of claim 31, wherein the collected data is stored in the blockchain based on a consensus mechanism ensuring that the collected data is verified and accurate.

33. The system of claim 30, wherein the collected data comprises one or more of: patient medical data; historical data; patient parameters; race; and previous diagnosis.

34. The system of claim 31, wherein the system comprises Internet of Things (IoT) devices writing records related to the patient directly to the blockchain.

35. The system of claim 30, wherein the machine learning models predict or diagnose the health of the patient that is associated with one or more of: depression/anxiety; mortality; readmission; and emergency department visits.

36. The system of claim 30, wherein the patient is a cardiovascular diseases (CVD) patient.

37. A system comprising:
an Internet-based cognitive behavioral therapy (iCBT) system collecting patient data associated with assessment and screening of a patient
a remote patient monitoring system collecting patient data associated with remote and real-time monitoring of the patient, wherein the remote and real-time monitoring comprises vital sign measurements, and further wherein the remote patient monitoring system comprises:
one or more data collection devices for obtaining vital sign measurements of the patient, wherein the one or more data collection devices comprises wearable devices;
a mobile device for obtaining the vital sign measurements of the patient using a video-based vital sign capture; and
a computer device communicatively connected to the one or more data collection devices and the mobile device to receive the obtained vital sign measurements of the patient, wherein the computer device enables the remote and real-time monitoring of the patient based on the received vital sign measurements of the patient
an artificial intelligent (AI) machine learning system collecting patient data associated with health diagnosis and predictions for the patient and
an integrated system receiving the patient from the iCBT system, the patient data from the remote patient monitoring system, and the patient data from the AI machine learning system, and analyzing the combination of the patient data to enable evidence-based interventions, diagnoses, and predictive health for the patient.

38. The system of claim 37, wherein the mobile device comprises a digital camera capturing video imaging data of the body of the patient, and analyzes the video imaging data using one or more optical analysis techniques to obtain the vital sign measurements of the patient using the video-based vital sign capture.

39. The system of claim 38, wherein the vital signs measurements of the patient obtained by the wearable devices and the mobile device comprise one or more of: heart rate; blood pressure; oxygen saturation (e.g., SpO₂); body temperature; pulse rate; respiration rate; and measurements of bodily functions monitored by medical professionals.

40. A system, comprising:
an Internet-based cognitive behavioral therapy (iCBT) system collecting patient data associated with assessment and screening of a patient
a remote patient monitoring system collecting patient data associated with remote and real-time monitoring of the patient, wherein the remote and real-time monitoring comprises vital sign measurements, and further wherein the remote patient monitoring system comprises:
one or more data collection devices for obtaining vital sign measurements of the patient, wherein the one or more data collection devices comprises wearable devices;
a mobile device for obtaining the vital sign measurements of the patient using a video-based vital sign capture; and
a computer device communicatively connected to the one or more data collection devices and the mobile device to receive the obtained vital sign measurements of the patient, wherein the computer device enables the remote and real-time monitoring of the patient based on the received vital sign measurements of the patient
an artificial intelligent (AI) machine learning system collecting patient data associated with health diagnosis and predictions for the patient and providing AI assisted remote patient monitoring, data collection, and data analysis, the AI machine learning system comprising:
a diagnostic server connected to the AI machine learning system via a network, the diagnostic server collecting data from one or more medical data silos; and
remote users connected to the diagnostic server via the network, wherein the remote users comprise doctors and patients, and wherein the AI machine learning system receives the collected data as training data, and trains machine learning models using the training data to generate the patient data associated with health diagnosis and predictions for the patient and
an integrated system receiving the patient from the iCBT system, the patient data from the remote patient monitoring system, and the patient data from the AI machine learning system, and analyzing the combination of the patient data to enable evidence-based interventions, diagnoses, and predictive health for the patient.
